(19) United States
(12) Patent Application Publication
(10) Pub. No.: US 2012/0136248 A1
(43) Pub. Date: May 31, 2012

(54) ULTRASOUND DIAGNOSIS APPARATUS, IMAGE GENERATING METHOD, AND IMAGE PROCESSING APPARATUS

(75) Inventors: Yuko Kanayama, Nasushiobara-shi (JP); Naohisa Kamiyama, Otawara-shi (JP)

(73) Assignees: TOSHIBA MEDICAL SYSTEMS CORPORATION, Otawara-shi (JP); KABUSHIKI KAISHA TOSHIBA, Tokyo (JP)

(21) Appl. No.: 13/303,470

(22) Filed: Nov. 23, 2011

(30) Foreign Application Priority Data
Nov. 25, 2010 (JP) 2010-262805

(51) Int. Cl. A61B 8/00; G06K 9/00

(52) U.S. Cl. 600/437; 382/131

(57) ABSTRACT

An ultrasound diagnosis apparatus according to an embodiment includes: a movement information obtaining unit that obtains movement information by adjusting a reception signal received by an ultrasound probe, by using a gain; an image generating unit that generates a color image in which color pixels are allocated based on the movement information; an index calculating unit that calculates an index value indicating a distribution of the color pixels, based on two or more of the color images generated by the image generating unit; a gain selecting unit that selects a display gain by comparing the index values calculated with respect to two or more of the gains; and a controlling unit that controls the image generating unit so as to generate a color image in which color pixels are allocated based on the display gain and the movement information obtained from the reception signal.

### CROSS-REFERENCE TO RELATED APPLICATIONS

This application is based upon and claims the benefit of priority of the prior Japanese Patent Application No. 2010-262805, filed on Nov. 25, 2010, the entire contents of which are incorporated herein by reference.

### FIELD

Embodiments described herein relate generally to an ultrasound diagnosis apparatus, an image generating method, and an image processing apparatus.

### BACKGROUND

Ultrasound diagnosis apparatuses have conventionally been considered to have more advantageous characteristics than other medical image diagnosis apparatuses such as X-ray diagnosis apparatuses and X-ray computed tomography apparatuses, due to the simple operability and the noninvasiveness (i.e., no possibility of causing radiation exposures). Ultrasound diagnosis apparatuses have been used in modern-day medicine for performing medical examinations and making diagnoses on various tissues of patients' bodies such as the heart, the liver, the kidney, and the mammary gland.

Ultrasound diagnosis apparatuses are configured so as to generate, in a real-time manner, a tomographic image (a Brightness-mode [B-mode] image) of a tissue structure of an examined subject (hereinafter, “patient”), by transmitting an ultrasound wave from an ultrasound probe and receiving a reflected-wave signal reflected by an internal tissue of the patient. Further, ultrasound diagnosis apparatuses in recent years are configured so as to generate, in a real-time manner, a color Doppler image that displays bloodstream information such as the speed, the dispersion, and the power of the bloodstream in such a manner that the bloodstream information is recognizable in colors, together with an area in which the bloodstream is present within the patient, by utilizing the Doppler effect of ultrasound waves. In addition, bloodstream information can quantitatively be analyzed by using such color Doppler images.

In some situations, however, the conventional ultrasound diagnosis apparatuses may generate mutually-different color Doppler images, depending on setting operations performed by one or more operators. More specifically, the ultrasound diagnosis apparatuses are configured so as to calculate the bloodstream information by using a Doppler shift frequency or the like of the ultrasound wave, after performing a gain adjusting process on a reception signal received by the ultrasound probe, by using a predetermined color gain. As a result, even if images of the same site of the same patient are taken, a conventional ultrasound diagnosis apparatus has a possibility of generating color Doppler images rendering mutually-different displays of colors, depending on the color gain that is set by the operator or the like.

### BRIEF DESCRIPTION OF THE DRAWINGS

FIG. 1 is a block diagram of an exemplary configuration of an ultrasound diagnosis apparatus according to a first embodiment;

FIG. 2 is a drawing of an exemplary color Doppler image;

FIG. 3 is a drawing of another exemplary color Doppler image;

FIG. 4 is a block diagram of an exemplary configuration of a controlling unit according to a first embodiment;

FIG. 5 is a drawing of an example of a pixel ratio storage unit;

FIG. 6 is a chart of an exemplary relationship between color gains and color pixel ratios;

FIG. 7 is a drawing of an example of a pixel number storage unit;

FIG. 8 is a chart of an exemplary relationship between a time lapse and the number of color pixels;

FIG. 9 is a flowchart of a processing procedure performed by the ultrasound diagnosis apparatus according to the first embodiment;

FIG. 10 is a flowchart of a procedure in an optimal gain selecting process performed by a gain selecting unit according to the first embodiment;

FIG. 11 is a flowchart of a procedure in an optimal image selecting process performed by an image selecting unit according to the first embodiment;

FIG. 12 is a chart of an exemplary relationship between color gains and color pixel ratios;

FIG. 13 is a block diagram of an exemplary configuration of a controlling unit according to a second embodiment;

FIG. 14 is a drawing of an example of a cutoff frequency storage unit;

FIG. 15 is a flowchart of a processing procedure performed by an ultrasound diagnosis apparatus according to the second embodiment;

FIG. 16 is a block diagram of an exemplary configuration of a controlling unit according to a third embodiment;

FIG. 17 is a drawing of an example of an optimal color gain storage unit;

FIG. 18 is a flowchart of a procedure in an optimal gain selecting process performed by an ultrasound diagnosis apparatus according to the third embodiment;

FIG. 19 is a block diagram of an exemplary configuration of a controlling unit according to a fourth embodiment;

FIG. 20 is a flowchart of a processing procedure performed by an ultrasound diagnosis apparatus according to the fourth embodiment;

FIG. 21 is a flowchart of a procedure in an optimal gain selecting process performed by a gain selecting unit according to the fourth embodiment; and

FIG. 22 is a diagram of an exemplary configuration of an image processing system.

### DETAILED DESCRIPTION

An ultrasound diagnosis apparatus according to an embodiment includes a movement information obtaining unit, an image generating unit, an index calculating unit, a gain selecting unit, and a controlling unit. The movement information obtaining unit obtains movement information by adjusting a reception signal received by an ultrasound probe, by using a gain. The image generating unit generates a color image in which color pixels are allocated based on the movement information. The index calculating unit calculates an index value indicating a distribution of the color pixels, based on two or more of the color images generated by the image generating unit. The gain selecting unit selects a display gain by comparing the index values calculated with respect to two or more of the gains. The controlling unit controls the image generating unit so as to generate a color image in which color pixels are allocated based on the display gain and the movement information obtained from the reception signal.

### First Embodiment

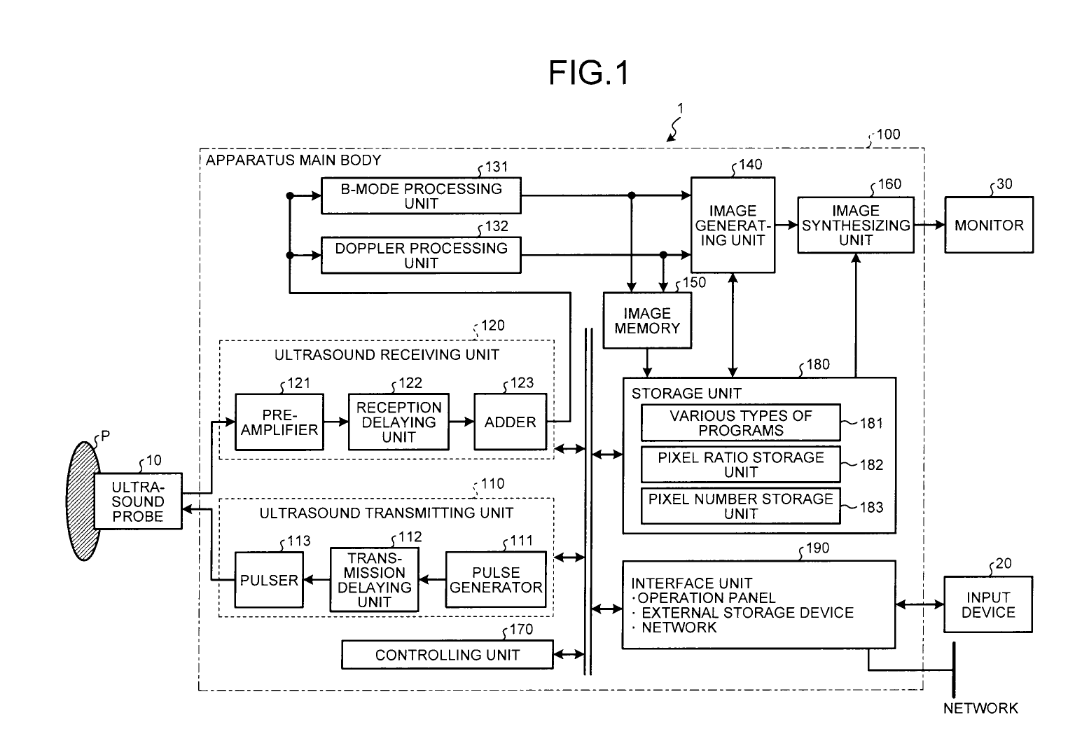

First, a configuration of an ultrasound diagnosis apparatus according to a first embodiment will be explained. FIG. 1 is a block diagram of an exemplary configuration of the ultrasound diagnosis apparatus according to the first embodiment. As illustrated in FIG. 1, an ultrasound diagnosis apparatus 1 according to the first embodiment includes an ultrasound probe 10, an input device 20, a monitor 30, and an apparatus main body 100.

The ultrasound probe 10 includes a plurality of piezoelectric vibrators. The plurality of piezoelectric vibrators generate an ultrasound wave based on a drive signal supplied by an ultrasound transmitting unit 110 included in the apparatus main body 100 (explained later). Further, the ultrasound probe 10 receives a reflected-wave signal from a patient P and converts the received reflected-wave signal into an electric signal. Further, the ultrasound probe 10 includes a matching layer with which the piezoelectric vibrators are provided, as well as a backing member that prevents the ultrasound wave from propagating rearward from the piezoelectric vibrators. The ultrasound probe 10 is detachably connected to the apparatus main body 100.

When the ultrasound wave is transmitted from the ultrasound probe 10 to the patient P, the transmitted ultrasound wave is repeatedly reflected on a surface of discontinuity of acoustic impedances at a tissue inside the body of the patient P and is received as the reflected-wave signal by the plurality of piezoelectric vibrators included in the ultrasound probe 10. The amplitude of the received reflected-wave signal is dependent on the difference between the acoustic impedances on the surface of discontinuity on which the ultrasound wave is reflected. When the transmitted ultrasound pulse is reflected on the surface of a flowing bloodstream or a cardiac wall, the reflected-wave signal is, due to the Doppler effect, subject to a frequency shift, depending on a velocity component of the moving member with respect to the ultrasound wave transmission direction.

The input device 20 is connected to the apparatus main body 100 and includes a mouse, a keyboard, a button, a panel switch, a touch command screen, a foot switch, a trackball, and the like. The input device 20 receives various types of setting requests from the operator of the ultrasound diagnosis apparatus 1 and transfers the received various types of setting requests to the apparatus main body 100. For example, the input device 20 receives a setting request to set a Region of Interest (ROI) or a setting request to set a color gain, from the operator.

The monitor 30 displays a Graphical User Interface (GUI) used by the operator of the ultrasound diagnosis apparatus 1 to input the various types of setting requests through the input device 20 and displays an ultrasound image generated by the apparatus main body 100. More specifically, the monitor 30 displays morphological information and bloodstream information within a patient's body as an image, based on a video signal input thereto from an image synthesizing unit 160 (explained later).

The apparatus main body 100 generates the ultrasound image based on the reflected-wave signal received by the ultrasound probe 10. As illustrated in FIG. 1, the apparatus main body 100 includes the ultrasound transmitting unit 110, an ultrasound receiving unit 120, a B-mode processing unit 131, a Doppler processing unit 132, an image generating unit 140, an image memory 150, the image synthesizing unit 160, a controlling unit 170, a storage unit 180, and an interface unit 190.

The ultrasound transmitting unit 110 includes a pulse generator 111, a transmission delaying unit 112, and a pulser 113 and supplies the drive signal to the ultrasound probe 10. The pulse generator 111 repeatedly generates a rate pulse for forming the transmission ultrasound wave at a predetermined rate frequency. Further, the transmission delaying unit 112 converges the ultrasound wave generated by the ultrasound probe 10 into the form of a beam and applies a delay period that is required to determine transmission directionality and that corresponds to each of the piezoelectric vibrators, to each of the rate pulses generated by the pulse generator 111. Further, the pulser 113 applies a drive signal (a drive pulse) to the ultrasound probe 10 with timing based on the rate pulses. Transmission directions and delay periods used for determining a transmission direction are stored in the storage unit 180. The transmission delaying unit 112 applies the delay periods by referring to the storage unit 180.

The ultrasound receiving unit 120 includes a preamplifier 121, an Analog/Digital (A/D) converter (not shown), a reception delaying unit 122, and an adder 123 and generates reflected-wave data by performing various types of processes on the reflected-wave signal received by the ultrasound probe 10. The preamplifier 121 amplifies the reflected-wave signal for each of channels. The A/D converter (not shown) applies an A/D conversion to the amplified reflected-wave signal. The reception delaying unit 122 applies a delay period that is required to determine reception directionality. The adder 123 generates the reflected-wave data by performing an adding process on the reflected-wave signal processed by the reception delaying unit 122. As a result of the adding process performed by the adder 123, reflected components from the direction corresponding to the reception directionality of the reflected-wave signal are emphasized, and an integrated beam for the ultrasound transmission and reception is formed, based on the reception directionality and the transmission directionality. Like in the example with the transmission, reception directions and delay periods used for determining a reception direction are stored in the storage unit 180. The reception delaying unit 122 applies the delay periods by referring to the storage unit 180.

The B-mode processing unit 131 receives the reflected-wave data from the ultrasound receiving unit 120 and generates data (B-mode data) in which the signal strength is expressed by a degree of brightness, by performing a logarithmic amplification, an envelop detection process, and the like.

The Doppler processing unit 132 performs a gain adjusting process on the reflected-wave data received from the ultrasound receiving unit 120, by using a predetermined color gain. The Doppler processing unit 132 then performs a frequency analysis to obtain speed information from the gain-adjusted reflected-wave data. Further, the Doppler processing unit 132 extracts the bloodstream, tissues, and contrast echo components by utilizing the Doppler effect, and calculates bloodstream information including an average speed, the dispersion, the power, and the like for a plurality of points. In other words, the Doppler processing unit 132 serves as a bloodstream obtaining unit that performs the gain adjusting process (an amplifying process) on the reception signal received by the ultrasound probe 10 by using the color gain and that obtains the bloodstream information based on the reception signal on which the gain adjusting process was performed.

The image generating unit 140 generates an ultrasound image from the B-mode data generated by the B-mode processing unit 131 and the bloodstream information generated by the Doppler processing unit 132 and stores the generated ultrasound image into the image memory 150 (explained later) or the storage unit 180.

More specifically, from the B-mode data, the image generating unit 140 generates the B-mode image in which the signal strength is expressed by a degree of brightness. Further, from the bloodstream information, the image generating unit 140 generates, as a color image, a color Doppler image that displays the speed and the dispersion of the bloodstream as well as a power component and the like indicating a blood flow volume or the like, in such a manner that these pieces of information are recognizable in colors.

Further, the image generating unit 140 converts (by performing a scan convert process) a scanning line signal sequence from an ultrasound scan into a scanning line signal sequence in a video format used by, for example, television and generates the ultrasound image (the B-mode image or the color Doppler image) serving as a displayed image.

The image memory 150 is a memory for storing therein the ultrasound image generated by the image generating unit 140 and images each generated by performing an image processing process on an ultrasound image. For example, after a diagnosis is made, the operator is able to acquire an image recorded during the medical examination out of the image memory 150. It is possible to play back each image in the manner of a still image or in the manner of moving pictures involving a plurality of images. Further, the image memory 150 stores therein, as necessary, an image brightness signal that passed the ultrasound receiving unit 120 as well as other raw data, images obtained via a network, and the like.

The image synthesizing unit 160 generates a synthesized image by synthesizing text information of various parameters, scale graduations, body marks, and the like with the ultrasound image generated by the image generating unit 140. The synthesized image generated by the image synthesizing unit 160 is displayed on the monitor 30.

The controlling unit 170 is a Central Processing Unit (CPU) realizing a function as an information processing apparatus (a computer) and exercises control over the entire processes performed by the ultrasound diagnosis apparatus 1. More specifically, based on various types of instructions and the setting requests input by the operator via the input device 20 and various types of computer programs and various types of setting information read from the storage unit 180, the controlling unit 170 controls processes performed by the ultrasound transmitting unit 110, the ultrasound receiving unit 120, the B-mode processing unit 131, the Doppler processing unit 132, the image generating unit 140, and the image synthesizing unit 160, and also, exercises control so that the ultrasound images and the like stored in the image memory 150 are displayed on the monitor 30.

The storage unit 180 stores therein various types of computer programs 181 used for the ultrasound transmission and reception, the image processing process, and the display process, as well as various types of data such as diagnosis information (e.g., IDs of patients, observations of medical doctors), diagnosis protocols, and various types of setting information. The various types of computer programs 181 may include a computer program describing a procedure to perform the same processes as the processes performed by the controlling unit 170.

Further, the storage unit 180 may be used, as necessary, for storing therein any of the ultrasound images stored in the image memory 150. The various types of data stored in the storage unit 180 may be transferred to any external peripheral device via the interface unit 190.

Further, the storage unit 180 includes a pixel ratio storage unit 182 and a pixel number storage unit 183 storing therein results calculated by the controlling unit 170, as explained later. The pixel ratio storage unit 182 and the pixel number storage unit 183 will be explained in detail later.

The interface unit 190 is an interface related to the input device 20, an operation panel, another external storage device (not shown), and a network. The data such as the ultrasound images obtained by the ultrasound diagnosis apparatus 1 can be transferred by the interface unit 190 to any other apparatus via a network.

The ultrasound transmitting unit 110, the ultrasound receiving unit 120, and the like that are included in the apparatus main body 100 may be configured by using hardware such as an integrated circuit or may be realized in the manner of software by using a computer program having modules.

The overall configuration of the ultrasound diagnosis apparatus 1 according to the first embodiment has been explained. When having received a request to start an image taking process on the patient P, the ultrasound diagnosis apparatus 1 configured as described above displays the B-mode image and/or the color Doppler image of the patient P. In this situation, the color gain used for generating the color Doppler image is set by the operator. The color gain is a coefficient used for performing a gain adjusting process (an amplifying process) on the reception signal received by the ultrasound probe. Performing the gain adjusting process on the reception signal based on the color gain makes it possible to accurately obtain the bloodstream information even if the reception signal is small. Accordingly, if the color gain is too small, the bloodstream information is not accurately displayed in the color Doppler image. On the contrary, if the color gain is too large, because noises are also amplified, the color Doppler image displays the noises. In this situation, the term “noises” refers to not only the noise components included in the signals transmitted and received by the ultrasound probe 10, but also noise components occurring in the internal circuits or the like of the ultrasound diagnosis apparatus 1.

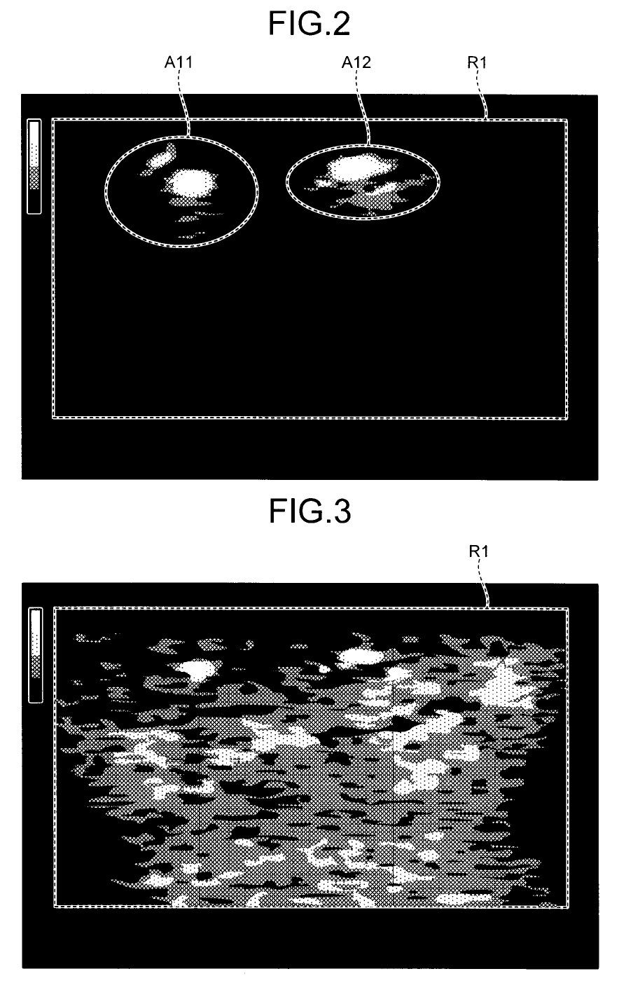

Next, examples of color Doppler images obtained using mutually-different color gains will be explained, with reference to FIGS. 2 and 3. FIGS. 2 and 3 are drawings of examples of color Doppler images. It is assumed that the color Doppler image shown in FIG. 2 is generated by using an optimal color gain by which the power, which is a type of bloodstream information, is accurately displayed. In contrast, it is assumed that the color Doppler image shown in FIG. 3 is generated by using a color gain that is larger than the color gain used for generating the color Doppler image shown in FIG. 2. The color Doppler images shown in FIGS. 2 and 3 are images of a predetermined region of interest R1 within mutually the same site of mutually the same patient P.

In the color Doppler image shown in FIG. 2, pixels to which colors are applied are present in areas A11 and A12, and the bloodstream information is accurately displayed. In contrast, in the color Doppler image shown in FIG. 3, colors are applied to the whole area, and the noises are displayed as color information.

As seen in these examples, even if images of mutually the same site of mutually the same patient P are taken, an ultrasound diagnosis apparatus may generate color Doppler images having mutually-different pieces of color information, when the color gains are set differently by the operator. Further, because the optimal value of the color gain is different for each of the different sites serving as image taking targets, it is not easy for the operator to always set an optimal color gain. For this reason, it is difficult for an ultrasound diagnosis apparatus to always use an optimal color gain. As a result, it may be considered difficult to always generate a color Doppler image in which the bloodstream information is accurately displayed.

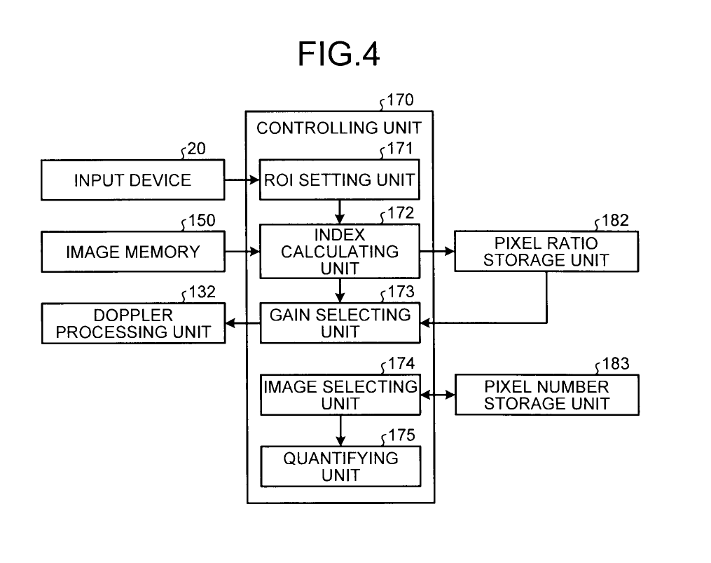

The ultrasound diagnosis apparatus 1 according to the first embodiment, however, makes it possible to set an optimal color gain as a result of the various types of processes performed by the controlling unit 170. In the following sections, the processes performed by the controlling unit 170 according to the first embodiment will primarily be explained, with reference to FIGS. 4 to 11.

First, a flow in a process performed by the controlling unit 170 according to the first embodiment will be explained. When having received a request from the operator to start an image taking process on the patient P, the controlling unit 170 according to the first embodiment first performs a process to set an optimal color gain. As a result, the controlling unit 170 makes it possible to generate a color Doppler image in which no noise or the like is displayed and in which the bloodstream information and the like are accurately displayed.

Further, after setting the optimal color gain and when having received a request from the operator indicating that an optimal color Doppler image should be selected, the controlling unit 170 selects an optimal color Doppler image in which no clutter component caused by the pulsation and/or body movements is displayed in colors, from among a plurality of color Doppler images generated for mutually the same image taking position. Further, the controlling unit 170 causes the optimal color Doppler image to be displayed on the monitor 30 and causes information that quantitatively indicates the bloodstream information contained in the optimal color Doppler image to be displayed on the monitor 30.

These processes described above performed by the controlling unit 170 will be explained in detail with reference to FIG. 4. FIG. 4 is a block diagram of an exemplary configuration of the controlling unit 170 according to the first embodiment. First, when having received a request from the operator to start an image taking process on the patient P, the controlling unit 170 causes a drive signal to be supplied to the ultrasound probe 10 by controlling the ultrasound transmitting unit 110.

Further, the controlling unit 170 instructs the Doppler processing unit 132 to perform a gain adjusting process on the reflected-wave data input thereto from the ultrasound receiving unit 120 by using a plurality of color gains that are different from each other by a predetermined difference. More specifically, the controlling unit 170 informs the Doppler processing unit 132 of a minimum value, a maximum value, and a change value of the color gains and instructs the Doppler processing unit 132 to calculate a plurality of pieces of bloodstream information from the same reflected-wave data, while sequentially changing the color gain by an amount corresponding to the change value, starting from the color gain minimum value.

As a result, by performing the gain adjusting process on the reflected-wave data input thereto from the ultrasound receiving unit 120 while using the plurality of color gains that are different from each other by the predetermined difference, the Doppler processing unit 132 according to the first embodiment calculates the bloodstream information corresponding to each of the color gains.

More specifically, the Doppler processing unit 132 stores the reflected-wave data input thereto from the ultrasound receiving unit 120 into a storage area such as the image memory 150 or the storage unit 180. Further, the Doppler processing unit 132 calculates bloodstream information from the reflected-wave data stored in the storage unit 180, by using the color gain maximum value informed by the controlling unit 170. Subsequently, the Doppler processing unit 132 adds the change value to the color gain minimum value and calculates bloodstream information from the reflected-wave data stored in the storage unit 180, by using the color gain resulting from the addition. In this manner, the Doppler processing unit 132 calculates the pieces of blood information while changing the color gain, until the color gain reaches the maximum value informed by the controlling unit 170.

Each of the pieces of bloodstream information is information indicating the speed, the dispersion, the power, and the like of the bloodstream. The Doppler processing unit 132 calculates the pieces of bloodstream information indicating the speed, the dispersion, the power, and/or the like of the bloodstream, depending on what display mode was selected by the operator from among a speed display mode, a dispersion display mode, a power display mode, or a display mode combining these pieces of information.

After that, the image generating unit 140 according to the first embodiment performs a process of generating color Doppler images from the pieces of bloodstream information calculated by the Doppler processing unit 132, for each of the pieces of bloodstream information that are calculated by the Doppler processing unit 132 and correspond to the mutually-different gains. In other words, from the plurality of pieces of bloodstream information calculated by using the mutually-different color gains, the image generating unit 140 generates the color Doppler images of which the quantity is equal to the number of pieces of bloodstream information.

Accordingly, for each of the color gains used by the Doppler processing unit 132 to generate the pieces of bloodstream information, the image generating unit 140 generates a color Doppler image corresponding to the color gain. The image generating unit 140 stores the plurality of color Doppler images generated in this manner into the image memory 150. Although the image generating unit 140 may store the color Doppler images into the storage unit 180, it is assumed that the image generating unit 140 stores the color Doppler image into the image memory 150 in the first embodiment.

After that, by analyzing the plurality of color Doppler images generated by the image generating unit 140, the controlling unit 170 determines the optimal color gain. More specifically, as illustrated in FIG. 4, the controlling unit 170 includes an ROI setting unit 171, an index calculating unit 172, and a gain selecting unit 173. As a result of processes performed by the index calculating unit 172 and the gain selecting unit 173, the controlling unit 170 determines the optimal color gain.

The ROI setting unit 171 sets a predetermined region of interest with respect to each of the color Doppler images that are color images generated by the image generating unit 140. More specifically, the ROI setting unit 171 receives, via the interface unit 190, a region-of-interest setting request received by the input device 20 and sets a region of interest in each of the color Doppler images according to the received region-of-interest setting request. For example, when the operator performed an operation to set a partial area of a color Doppler image as a region of interest, it is possible to reduce the processing loads because the index calculating unit 172 and the gain selecting unit 173 (explained later) use only the partial area as a processing target.

The index calculating unit 172 calculates an index value indicating a distribution of each of the color Doppler images, based on the plurality of color Doppler images generated by the image generating unit 140. The index calculating unit 172 according to the first embodiment calculates, as the index value, the number of pixels to which colors are applied within the region of interest set by the ROI setting unit 171, for each of the color Doppler images generated by the image generating unit 140. In the following sections, pixels to which colors are applied will be referred to as “color pixels”.

Next, the process performed by the index calculating unit 172 will be explained more specifically. When the image generating unit 140 has generated the color Doppler images respectively corresponding to the mutually-different color gains, the index calculating unit 172 sequentially obtains the color Doppler images from the memory 150. After that, the index calculating unit 172 counts the number of color pixels contained in the region of interest in each of the color Doppler images obtained from the image memory 150. Further, the index calculating unit 172 calculates a “color pixel ratio” indicating the ratio between the counted number of color pixels and the total number of pixels within the region of interest. For example, the index calculating unit 172 calculates the color pixel ratio by dividing the counted number of color pixels by the total number of pixels within the region of interest.

The index calculating unit 172 calculates the color pixel ratio for the region of interest that is set in mutually the same position among the plurality of color Doppler images stored in the image memory 150. Further, the index calculating unit 172 stores the color gains used for generating the color Doppler images and the color pixel ratios of the color Doppler images, which are kept in correspondence with one another, into the pixel ratio storage unit 182.

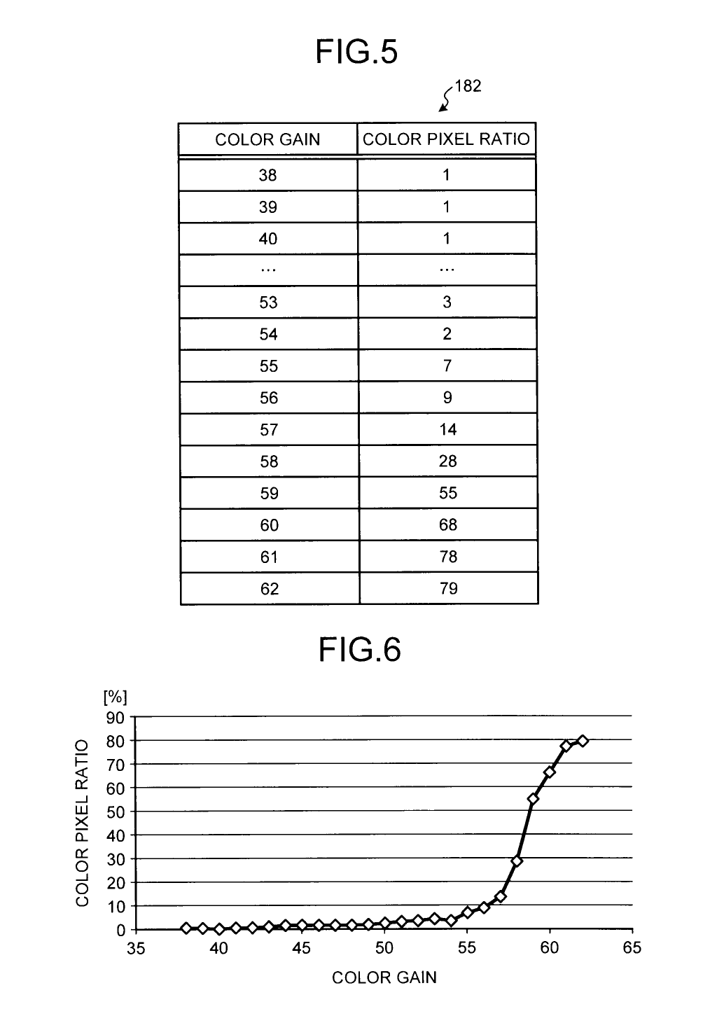

Next, the pixel ratio storage unit 182 will be explained, with reference to FIG. 5. FIG. 5 is a drawing of an example of the pixel ratio storage unit 182. As shown in FIG. 5, the pixel ratio storage unit 182 includes items such as “color gain” and “color pixel ratio”.

The item “color gain” denotes each of the color gains used for generating the color Doppler images. More specifically, the “color gain” denotes each of the color gains used by the Doppler processing unit 132 to calculate the bloodstream information from which the color Doppler images are generated. The item “color pixel ratio” denotes the color pixel ratio of the color Doppler image generated from the bloodstream information calculated by using the corresponding color gain. In the first embodiment, the “color pixel ratio” denotes the color pixel ratio of the region of interest in the color Doppler image.

For example, the pixel ratio storage unit 182 shown in FIG. 5 indicates that the color pixel ratio of the color Doppler image generated from the bloodstream information calculated by using the color gain “38” is “1%”. As another example, the pixel ratio storage unit 182 shown in FIG. 5 indicates that the color pixel ratio of the color Doppler image corresponding to the color gain “53” is “3%”.

The example shown in FIG. 5 indicates that the controlling unit 170 informed the Doppler processing unit 132 of the color gain minimum value “38”, the color gain maximum value “62”, and the change value “1”. In other words, in the example shown in FIG. 5, the Doppler processing unit 132 calculates the bloodstream information by using the color gain minimum value “38” and keeps calculating the bloodstream information while increasing the color gain by the change value “1” until the color gain reaches the color gain maximum value “62”.

Returning to the description of FIG. 4, the gain selecting unit 173 selects a display-purpose color gain, by comparing the index values calculated with respect to the plurality of color gains. More specifically, the gain selecting unit 173 according to the first embodiment extracts color pixel ratios of two color Doppler images corresponding to two adjacent color gains, from among the color pixel ratios calculated by the index calculating unit 172. After that, the gain selecting unit 173 selects the smallest value of a group made up of the color gains by which the change amount between the two extracted color pixel ratios is equal to or larger than a predetermined change-amount threshold value, as an optimal gain.

More specifically, the gain selecting unit 173 obtains two color pixel ratios corresponding to two adjacent color gains from the pixel ratio storage unit 182 updated by the index calculating unit 172 and judges whether the change amount between the two obtained color pixel ratios is equal to or larger than the predetermined change-amount threshold value. In this situation, the gain selecting unit 173 sequentially obtains two color pixel ratios corresponding to two adjacent color gains, starting from the color gain minimum value. When the change amount between the color pixel ratios is equal to or larger than the change-amount threshold value, the gain selecting unit 173 selects the smaller of the two color gains as the optimal color gain.

For example, let us assume that the various types of data stored in the pixel ratio storage unit 182 are in the state shown in FIG. 5. In the present example, the change-amount threshold value is assumed to be “5”. In this situation, the gain selecting unit 173 first extracts the two adjacent color gains “38” and “39” from the pixel ratio storage unit 182. Further, the gain selecting unit 173 calculates the change amount between the color pixel ratio “1” corresponding to the extracted color gain “38” and the pixel ratio “1” corresponding to the color gain “39” as “0 (=1−1)”.

After that, the gain selecting unit 173 judges whether the calculated change amount “0” is equal to or larger than the change-amount threshold value “5”. In this situation, the gain selecting unit 173 determines that the change amount “0” is smaller than the change-amount threshold value “5”.

Subsequently, the gain selecting unit 173 extracts the two adjacent color gains “39” and “40” from the pixel ratio storage unit 182. Further, the gain selecting unit 173 calculates the change amount between the color pixel ratio “1” corresponding to the extracted color gain “39” and the color pixel ratio “1” corresponding to the color gain “40” as “0 (=1−1)”. After that, the gain selecting unit 173 judges whether the calculated change amount “0” is equal to or larger than the change-amount threshold value “5”.

The gain selecting unit 173 performs the process described above until the change amount between the color pixel ratios becomes equal to or larger than the change-amount threshold value. In the example shown in FIG. 5, the gain selecting unit 173 determines that the calculated change amount “5 (=7−2)” between the color pixel ratios is equal to or larger than the change-amount threshold value “5”, when extracting the two adjacent color gains “54” and “55”.

In that situation, the gain selecting unit 173 selects the smaller of the two color gains by which the change amount between the color pixel ratios is equal to or larger than the change-amount threshold value, as an optimal color gain. In the example described above, because the gain selecting unit 173 determines that the change amount between the color pixel ratios is equal to or larger than the change-amount threshold value when having extracted the color gains “54” and “55”, the gain selecting unit 173 selects the color gain “54” as the optimal color gain.

In the following sections, the reason why it is possible to select the optimal color gain through the process performed by the gain selecting unit 173 described above will be explained. First of all, ultrasound diagnosis apparatuses have a characteristic where, when the color gain is gradually increased, the amount of noises contained in the color Doppler image radically increases at a predetermined color gain serving as a border line. This characteristic will be explained with reference to the example shown in FIG. 6. FIG. 6 is a chart of an exemplary relationship between color gains and color pixel ratios. The chart in FIG. 6 expresses the relationship between the color gains and the color pixel ratios stored in the pixel ratio storage unit 182 illustrated in FIG. 5.

Generally speaking, as observed in the example in FIG. 6, when the color gain is small, the color pixel ratio is low. In contrast, when the color gain is large, the color pixel ratio is high. Further, as observed in the example in FIG. 6, when the color gain is gradually increased, the color pixel ratio of the color Doppler image radically increases, when the color gain exceeds the predetermined value. This is because the proportion of the noises contained in the color Doppler image increases, when the color gain is equal to or larger than the predetermined value.

In the example in FIG. 6, the change amounts in the color pixel ratios are small, and also, the color pixel ratios are low, when the color gain is in the range from “38” to “54”. In other words, while the color gain is in the range from “38” to “54”, almost no noise is displayed in the color Doppler image. In contrast, when the color gain is “55” or larger, the color pixel ratio radically increases. In other words, when the color gain is “55” or larger, the amount of noises displayed in the color Doppler image radically increases. Accordingly, when the color gain is gradually changed, the color gain at which the color pixel ratio starts increasing radically is considered to be the border line between the color gain by which almost no noise is displayed in the color Doppler image and the color gain by which noises are displayed in the color Doppler image.

In this situation, the optimal color gain refers to the gain by which it is possible to generate a color Doppler image in which as little noise as possible is contained and in which the bloodstream information is accurately displayed. As explained with reference to FIG. 3, noises are displayed in the color Doppler image if the color gain is too large, whereas the bloodstream information is not accurately displayed in the color Doppler image if the color gain is too small, because the bloodstream information is thinned out. Accordingly, the optimal color gain is the largest value among the color gains by which almost no noise is displayed in the color Doppler image.

For this reason, the gain selecting unit 173 according to the first embodiment identifies the color gain at which the color pixel ratio starts increasing radically, based on the various types of data contained in the pixel ratio storage unit 182. Further, the gain selecting unit 173 selects the largest value from among the color gains smaller than the identified color gain, as the optimal color gain. In the example in FIG. 6, the gain selecting unit 173 identifies the color gain “55” as the color gain at which the color pixel ratio starts increasing radically. After that, the gain selecting unit 173 selects the largest value “54” from among the color gains smaller than the color gain “55”, as the optimal color gain.

The index calculating unit 172 and the gain selecting unit 173 select the optimal color gain in this manner. Further, the controlling unit 170 configures the color gain selected by the gain selecting unit 173 into the Doppler processing unit 132. More specifically, the controlling unit 170 controls the Doppler processing unit 132 so as to use the color gain selected by the gain selecting unit 173. For example, when the Doppler processing unit 132 is configured so as to use the color gain stored in the storage unit 180, the controlling unit 170 stores the color gain selected by the gain selecting unit 173 into the storage unit 180. As a result, the Doppler processing unit 132 is able to calculate the bloodstream information by using the optimal color gain, and the image generating unit 140 is able to generate a color Doppler image in which almost no noise is contained and in which the bloodstream information is accurately displayed.

The gain selecting unit 173 may display the relationship between the color gains and the color pixel ratios illustrated in FIG. 6 on the display monitor 30 or may store the relationship into the storage unit 180. With this arrangement, the ultrasound diagnosis apparatus 1 is able to have the operator confirm whether the color gain is appropriately set, when the operator browses the color Doppler image later, for example.

After the optimal color gain is set in this manner, the ultrasound diagnosis apparatus 1 generates a color Doppler image by using the optimal color gain. In this situation, when having received a request from the operator indicating that a color Doppler image having an optimal temporal phase should be displayed, the controlling unit 170 according to the first embodiment selects a color Doppler image having a small amount of clutter components from among the plurality of color Doppler images generated for mutually the same image taking position and causes the selected color Doppler image to be displayed on the monitor 30. Further, according to a request from the operator, the controlling unit 170 calculates quantitative information that quantitatively indicates the bloodstream information contained in the optimal color Doppler image and causes the calculated quantitative information to be displayed on the monitor 30.

More specifically, as illustrated in FIG. 4, the controlling unit 170 includes an image selecting unit 174 and a quantifying unit 175. The controlling unit 170 performs a process to select the optimal color Doppler image through a process performed by the image selecting unit 174, and performs a process to calculate the quantitative information through a process performed by the quantifying unit 175.

The image selecting unit 174 selects a color Doppler image of which the number of color pixels is either the smallest value or the largest value as an optimal color Doppler image, from the image memory 150 storing therein the plurality of color Doppler images generated for mutually the same image taking position. In this situation, the operator is able to perform a setting operation to select a color Doppler image of which either the number of color pixels is the smallest value or the number of color pixels is the largest value. According to the setting operation performed by the operator, the image selecting unit 174 determines which of the color Doppler images should be selected, the color Doppler image of which the number of color pixels is the smallest value or the largest value. In the following sections, the process performed by the image selecting unit 174 will be explained more specifically.

First, because the ultrasound diagnosis apparatus 1 is configured so as to be able to display the bloodstream information of the patient P on the monitor 30 in a real-time manner, the image generating unit 140 is configured, in many examples, so as to generate one color Doppler image for every predetermined time period, for the same image taking position. As a result, the image memory 150 stores therein a plurality of color Doppler images that are generated for mutually the same image taking position and have mutually-different temporal phases (i.e., mutually-different image taking times).

Accordingly, when having received a request from the operator indicating that an optimal color Doppler image should be displayed, the image selecting unit 174 obtains the plurality of color Doppler images having the mutually-different temporal phases from the image memory 150. Further, the image selecting unit 174 counts the number of color pixels for each of the plurality of color Doppler images obtained from the image memory 150. In other words, the image selecting unit 174 counts the number of color pixels of each of the plurality of color Doppler images that are stored in the image memory 150 and are generated for mutually the same image taking position. Further, the image selecting unit 174 stores pieces of image identification information identifying the color Doppler images and the numbers of color pixels of the color Doppler images, which are kept in correspondence with one another, into the pixel number storage unit 183.

In this situation, the image selecting unit 174 does not necessarily have to count the number of color pixels for each of all the plurality of color Doppler images generated for mutually the same image taking position. For example, the image selecting unit 174 may count the number of color pixels for each of a predetermined number of color Doppler images. Alternatively, the image selecting unit 174 may count the number of color pixels, for example, within a region of interest set by the ROI setting unit 171 in the color Doppler images.

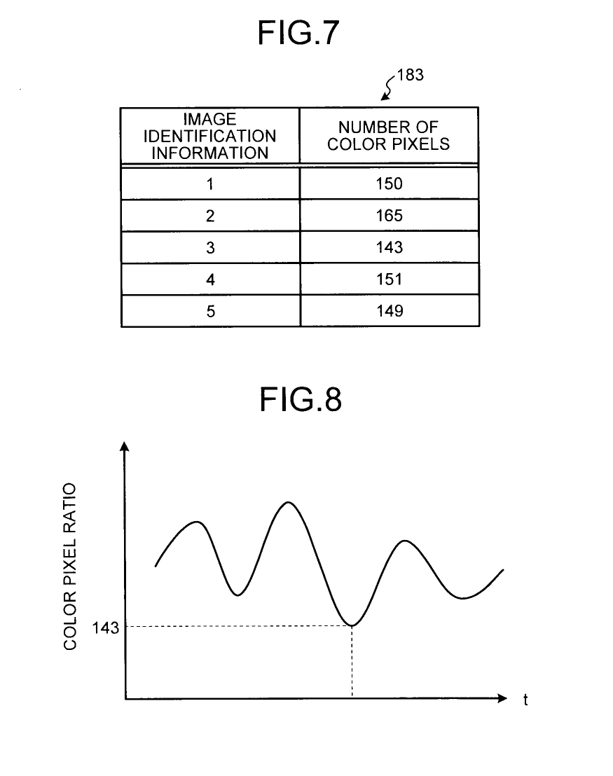

Next, the pixel number storage unit 183 will be explained, with reference to FIG. 7. FIG. 7 is a drawing of an example of the pixel number storage unit 183. As shown in FIG. 7, the pixel number storage unit 183 includes items such as “image identification information” and “number of color pixels”.

The item “image identification information” denotes information for identifying each of the color Doppler images. More specifically, the “image identification information” is information for identifying each of the color Doppler images that are generated for mutually the same image taking position and are stored in the image memory 150. The item “number of color pixels” denotes the number of color pixels of the color Doppler image identified by the corresponding piece of image identification information.

For example, the pixel number storage unit 183 shown in FIG. 7 indicates that the number of color pixels of the color Doppler image identified by the image identification information “1” is “150”. As another example, the pixel number storage unit 183 shown in FIG. 7 indicates that the number of color pixels of the color Doppler image identified by the image identification information “2” is “165”.

When having stored the various types of data into the pixel number storage unit 183 in this manner, the image selecting unit 174 selects a color Doppler image of which the number of color pixels stored in the pixel number storage unit 183 is either the smallest value or the largest value, as an optimal color Doppler image. To explain more specifically with reference to the example in FIG. 7, the image selecting unit 174 may select the color Doppler image identified by the image identification information “3” corresponding to the smallest number of color pixels “143” as an optimal color Doppler image, from among the numbers of color pixels “150”, “165”, “143”, “151” and “149” stored in the pixel number storage unit 183. Alternatively, the image selecting unit 174 may select the color Doppler image identified by the image identification information “2” corresponding to the largest number of color pixels “165” as an optimal color Doppler image.

After that, the image selecting unit 174 displays the selected optimal color Doppler image on the monitor 30. Further, according to an instruction from the operator, the image selecting unit 174 may store the selected optimal color Doppler image into the image memory 150 or the storage unit 180.

In the following sections, the reason why it is possible to select the optimal color Doppler image through the process performed by the image selecting unit 174 described above will be explained. As explained above, the Doppler processing unit 132 and the image generating unit 140 generate the plurality of color Doppler image for mutually the same image taking position, by using the optimal color gain selected by the gain selecting unit 173. Accordingly, in the plurality of color Doppler images generated in this manner, almost no noise is supposed to be displayed, and also, the bloodstream information and the like are supposed to be accurately displayed. In addition, because the color gains used during the generating process are mutually the same, the plurality of color Doppler images may be considered to be identical to one another.

However, because the plurality of color Doppler images generated by the image generating unit 140 have the mutually-different temporal phases, the color Doppler images show the tissue in the patient's body in the mutually-different temporal phases. Accordingly, in one or more of the plurality of color Doppler images, clutter components caused by the pulsation, body movements, and the like may be displayed as color information. Also, because changes occur in the bloodstream over the course of time (hereinafter “temporal changes”), not all of the plurality of color Doppler images are necessarily identical. Thus, it is considered that the number of color pixels changes between the color Doppler images generated by using the optimal color gain, due to the clutter components caused by the pulsation, body movements, and the like and the changes occurring in the bloodstream.

In this situation, when the tissue in the patient's body at the image taking site is a tissue having a small temporal change in the blood flow volume, it is considered that the number of color pixels changes between a plurality of color Doppler images primarily due to the clutter components. More specifically, as for the numbers of color pixels of the color Doppler images of the tissue in the patient's body having a small temporal change in the blood flow volume, the number of color pixels increases when many clutter components are contained and does not increase when no clutter components are contained. In other words, the smaller the number of color pixels present in a color Doppler image is, the fewer clutter components are contained in the color Doppler image. Accordingly, when the tissue in the patient's body at the image taking site is a tissue having a small temporal change in the blood flow volume, the image selecting unit 174 according to the first embodiment may select the color Doppler image of which the number of color pixels is the smallest value, from among the plurality of color Doppler images generated by the image generating unit 140 for mutually the same image taking position, as an optimal color Doppler image in which almost no clutter component is contained.

On the contrary, when the tissue in the patient's body at the image taking site is a tissue having a large temporal change in the blood flow volume (e.g., in an arterial phase), it is considered that the number of color pixels significantly changes between a plurality of color Doppler images due to the blood flow volume. Thus, when the bloodstream is in an arterial phase, it may be a good idea, in some situations, to select a color Doppler image in a temporal phase exhibiting the largest blood flow volume as an optimal image, because, for example, a large blood flow volume is expressed therein. Accordingly, when the tissue in the patient's body at the image taking site is a tissue having a large temporal change in the blood flow volume, the image selecting unit 174 according to the first embodiment may select the color Doppler image of which the number of color pixels is the largest value, from among the plurality of color Doppler images generated by the image generating unit 140 for mutually the same image taking position, as an optimal color Doppler image in which almost no clutter component is contained.

As explained above, according to the setting operation performed by the operator, the image selecting unit 174 selects the color Doppler image of which the number of color pixels is either the smallest value or the largest value, as an optimal image. In this situation, the image selecting unit 174 may calculate a change amount in the number of color pixels among the plurality of color Doppler images generated for mutually the same image taking position and may judge the temporal changes in the blood flow volume of the tissue in the patient's body at the image taking site based on the calculated change amount. Further, when the temporal changes in the blood flow volume are small, the image selecting unit 174 may select the color Doppler image of which the number of color pixels is the smallest value, as an optimal color Doppler image. On the contrary, when the temporal changes in the blood flow volume are large, the image selecting unit 174 may select the color Doppler image of which the number of color pixels is the largest value, as an optimal color Doppler image.

The explanation will be continued with reference to the example in FIG. 8. FIG. 8 is a chart of an exemplary relationship between a time lapse and the number of color pixels. In FIG. 8, the number of color pixels stored in the pixel number storage unit 183 shown in FIG. 7 is expressed in the chart.

As shown in the example in FIG. 8, even if the plurality of color Doppler images are generated for mutually the same image taking position by using the optimal color gain, the number of color pixels changes between the color Doppler images. The changes in the number of color pixels are considered to be, as explained above, the changes in the clutter components. In the first embodiment, because the color Doppler images are generated by using the optimal color gain, it is considered that, the larger the number of color pixels is in the color Doppler image, the more clutter components are contained therein. In other words, the smaller the number of color pixels is in the color Doppler image, the fewer clutter components are contained therein. For this reason, in the example in FIG. 8, the image selecting unit 174 selects the color Doppler image of which the number of color pixels is “143” as an optimal color Doppler image.

Returning to the description of FIG. 4, the quantifying unit 175 calculates quantitative information that quantitatively indicates the bloodstream information contained in the optimal color Doppler image selected by the image selecting unit 174. More specifically, a region of interest (hereinafter, “quantification region of interest”) used when the quantifying unit 175 calculates the quantitative information is specified in advance. Further, according to an instruction from the operator, the quantifying unit 175 calculates a ratio between the number of color pixels within the quantification region of interest in the optimal color Doppler image selected by the image selecting unit 174 and the total number of pixels within the quantification region of interest, as the quantitative information. Further, the quantifying unit 175 displays the calculated quantitative information on the monitor 30 or stores the calculated quantitative information into the storage unit 180.

In this situation, the quantifying unit 175 may calculate the number of color pixels of the entire color Doppler image or may calculate the color pixel ratio of the entire color Doppler image, as the quantitative information. Alternatively, the quantifying unit 175 may calculate a sum of the power values with respect to the color pixels within the quantification region of interest in the color Doppler image, as the quantitative information. As a yet another example, the quantifying unit 175 may calculate one or more selected out of the following as the quantitative information: the number of color pixels, the color pixel ratio, and a sum of the power values.

The quantitative information calculated by the quantifying unit 175 in this manner has a high reliability. For example, if a color gain is set by the operator, there is a possibility that the color Doppler image may contain noises. Thus, the quantitative information calculated from such a color Doppler image has a low reliability. Also, even if the color gain is set appropriately, because there is a possibility that the color Doppler image contains clutter components, the quantitative information calculated from such a color Doppler image has a low reliability. In other words, it is not possible to use the quantitative information calculated from a color Doppler image containing noises and/or clutter components, as an index for the bloodstream information.

In contrast, because the color Doppler image selected by the image selecting unit 174 does not contain noises or clutter components, the quantitative information calculated by the quantifying unit 175 has a high reliability. In other words, the quantifying unit 175 is able to provide the operator with the quantitative information that can serve as an index for the bloodstream information.

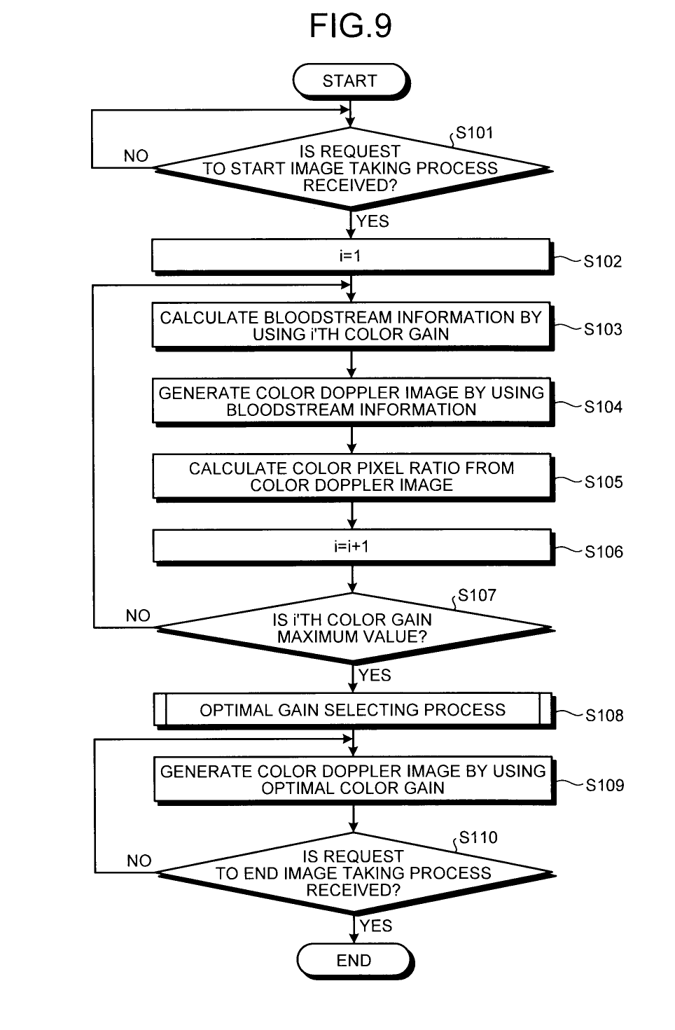

Next, a procedure in a process performed by the ultrasound diagnosis apparatus 1 according to the first embodiment will be explained, with reference to FIG. 9. FIG. 9 is a flowchart of a processing procedure performed by the ultrasound diagnosis apparatus 1 according to the first embodiment.

As shown in FIG. 9, the ultrasound diagnosis apparatus 1 according to the first embodiment judges whether a request to start an image taking process is received from the operator (step S101). If no request to start an image taking process is received (step S101: No), the ultrasound diagnosis apparatus 1 goes into a stand-by state.

On the contrary, if a request to start an image taking process is received (step S101: Yes), the controlling unit 170 sets a counter i to “1” (step S102). After that, under the control of the controlling unit 170, the Doppler processing unit 132 calculates bloodstream information by performing a gain adjusting process on reflected-wave data input from the ultrasound receiving unit 120, by using an i'th color gain (step S103). In this situation, the “i'th color gain” denotes a value obtained by adding the change value “i−1” times to the color gain minimum value of which the Doppler processing unit 132 is informed by the controlling unit 170.

Further, the image generating unit 140 generates a color Doppler image from the bloodstream information calculated by the Doppler processing unit 132 (step S104). After that, the index calculating unit 172 included in the controlling unit 170 calculates a color pixel ratio of the color Doppler image generated by the image generating unit 140 (step S105). For example, the index calculating unit 172 calculates the color pixel ratio by counting the number of color pixels within a predetermined region of interest in the color Doppler image and dividing the number of color pixels by the total number of pixels in the region of interest.

After that, the controlling unit 170 adds “1” to the counter i (step S106) and judges whether the i'th color gain is larger than the color gain maximum value of which the Doppler processing unit 132 is informed (step S107). In this situation, if the i'th color gain is equal to or smaller than the color gain maximum value (step S107: No), the process returns to step S103.

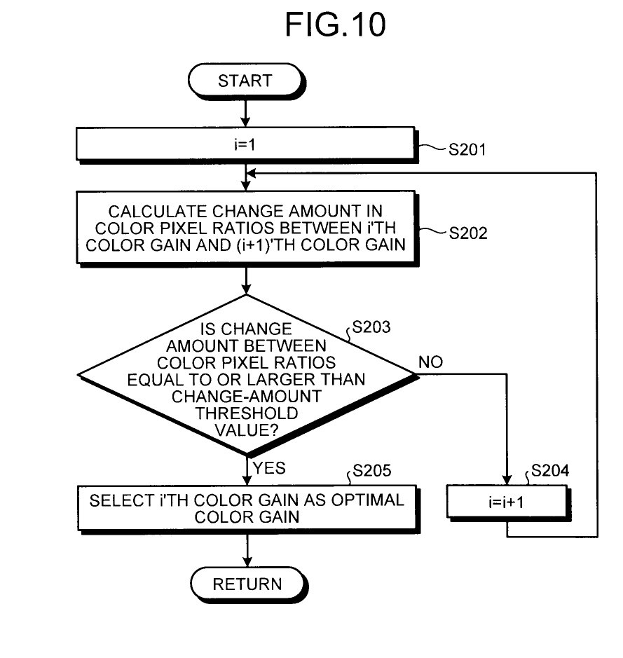

On the contrary, if the i'th color gain is larger than the color gain maximum value (step S107: Yes), the gain selecting unit 173 performs an optimal gain selecting process (step S108). The optimal gain selecting process performed by the gain selecting unit 173 will be explained in detail with reference to FIG. 10.

After that, the Doppler processing unit 132, the image generating unit 140, and the like generate a color Doppler image by using the optimal color gain selected by the gain selecting unit 173 (step S109). Further, the controlling unit 170 judges whether a request to end the image taking process is received from the operator (step S110). If no request to end the image taking process is received (step S110: No), the process returns to step S109. On the contrary, if a request to end the image taking process is received (step S110: Yes), the controlling unit 170 ends the process.

Next, a procedure in the optimal gain selecting process at step S108 in FIG. 9 will be explained, with reference to FIG. 10. FIG. 10 is a flowchart of the procedure in the optimal gain selecting process performed by the gain selecting unit 173 according to the first embodiment.

As shown in FIG. 10, the gain selecting unit 173 sets the counter i to “1” (step S201). After that, the gain selecting unit 173 obtains the color pixel ratio corresponding to the i'th color gain and the color pixel ratio corresponding to the (i+1)'th color gain from the pixel ratio storage unit 182 and calculates the change amount between the two obtained color pixel ratios (step S202).

After that, the gain selecting unit 173 judges whether the change amount between the color pixel ratios is equal to or larger than the change-amount threshold value (step S203). In this situation, if the change amount between the color pixel ratios is smaller than the change-amount threshold value (step S203: No), the gain selecting unit 173 adds “1” to the counter i (step S204), and the process returns to step S202.

On the contrary, if the change amount between the color pixel ratios is equal to or larger than the change-amount threshold value (step S203: Yes), the gain selecting unit 173 selects the i'th color gain as an optimal color gain (step S205).

Although not shown in FIG. 10, if there is no change amount between the color pixel ratios that is equal to or larger than the change-amount threshold value, the gain selecting unit 173 may, for example, display information on the monitor 30 indicating that it is not possible to select an optimal color gain. Alternatively, the gain selecting unit 173 may select the largest color gain among the plurality of color gains used by the Doppler processing unit 132, as an optimal color gain.

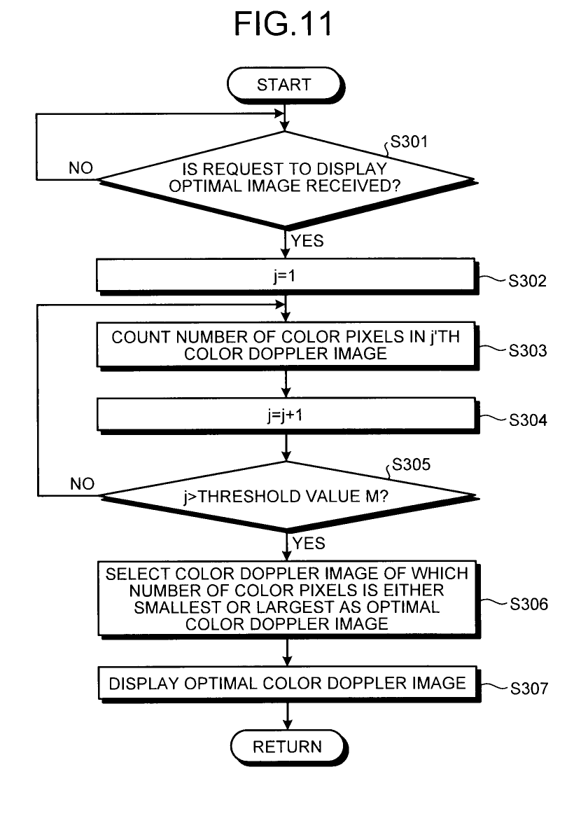

Next, a procedure in the optimal image selecting process performed by the image selecting unit 174 according to the first embodiment will be explained, with reference to FIG. 11. FIG. 11 is a flowchart of the procedure in the optimal image selecting process performed by the image selecting unit 174 according to the first embodiment.

As shown in FIG. 11, when having received a request from the operator indicating that an optimal color Doppler image should be displayed (step S301: Yes), the image selecting unit 174 sets a counter j to “1” (step S302).

After that, from the image memory 150 storing therein a plurality of color Doppler images generated for mutually the same image taking position, the image selecting unit 174 obtains a j'th color Doppler image and counts the number of color pixels of the obtained color Doppler image (step S303). In this situation, the image selecting unit 174 stores image identification information identifying the color Doppler image and the number of color pixels of the color Doppler image, which are kept in correspondence with each other, into the pixel number storage unit 183.

Further, the image selecting unit 174 adds “1” to the counter j (step S304) and judges whether the counter j is larger than a threshold value M (step S305). In this situation, if the counter j is equal to or smaller than the threshold value M (step S305: No), the image selecting unit 174 returns to step S303. In this situation, the “threshold value M” is the number of color Doppler images that are generated for mutually the same image taking position and are stored in the image memory 150.

On the contrary, if the counter j is larger than the threshold value M (step S305: Yes), the image selecting unit 174 selects the color Doppler image of which the number of color pixels stored in the pixel number storage unit 183 is either the smallest value or the largest value, as an optimal color Doppler image (step S306). After that, the image selecting unit 174 displays the optimal color Doppler image on the monitor 30 (step S307).

After that, if the operator requests quantitative information of the bloodstream information, the quantifying unit 175 calculates quantitative information such as the number of color pixels, the color pixel ratio, and/or a sum of the power values, based on the optimal Doppler image, and displays the calculated quantitative information on the monitor 30 or stores the calculated quantitative information into the storage unit 180.

As explained above, according to the first embodiment, the Doppler processing unit 132 calculates, for each of the color gains, the bloodstream information of the tissue in the patient's body in the image taking position, by performing the gain adjusting process on the reception signal received by the ultrasound probe 10 for mutually the same image taking position, by using the mutually-different color gains. Further, with respect to each of the pieces of bloodstream information respectively corresponding to the color gains, the image generating unit 140 performs the process of generating the color Doppler image to which different colors are applied depending on the bloodstream information, based on the pieces of bloodstream information calculated by the Doppler processing unit 132. Further, the index calculating unit 172 counts the number of color pixels for each of the color Doppler images generated by the image generating unit 140. Subsequently, the gain selecting unit 173 extracts the numbers of color pixels of the two color Doppler images corresponding to two adjacent color gains from among the numbers of color pixels counted by the index calculating unit 172 and selects the smallest value of the group made up of the color gains by which the change amount between the two extracted numbers of color pixels is equal to or larger than the change-amount threshold value, as an optimal color gain. After that, the controlling unit 170 controls the image generating unit 140 and the like so as to generate a color Doppler image by using the reflected-wave signal from the tissue in the patient's body on which the gain adjusting process is performed while using the color gain selected by the gain selecting unit 173.

As a result, the ultrasound diagnosis apparatus 1 according to the first embodiment is able to set the optimal color gain. In other words, the ultrasound diagnosis apparatus 1 is able to generate the color Doppler image in which as little noise as possible is contained and in which the bloodstream information is accurately displayed. More specifically, when the color gain is set by the operator, it is not necessarily always possible to generate a color Doppler image in which no noise is contained and in which the bloodstream information is accurately displayed, because the setting by the operator varies. In contrast, the ultrasound diagnosis apparatus 1 according to the first embodiment is able to generate, without fail, the color Doppler image in which no noise is contained and in which the bloodstream information is accurately displayed.

Further, the ultrasound diagnosis apparatus 1 according to the first embodiment transmits the ultrasound wave to the patient and sets the optimal color gain based on the reflected-wave signal from the patient. Thus, the ultrasound diagnosis apparatus 1 is able to set a color gain that is optimal for the image taking target site and for the environment in which the image taking process is performed.

Further, according to the first embodiment, the image generating unit 140 is controlled by the controlling unit 170 so as to perform the image generating process by using the optimal color gain and stores the plurality of color Doppler images generated for mutually the same image taking position into the image memory 150. After that, the image selecting unit 174 selects the color Doppler image of which the number of color pixels is the smallest value as an optimal color Doppler image, from among the plurality of color Doppler images that are generated for mutually the same image taking position and are stored in the image memory 150, and subsequently displays the selected color Doppler image on the monitor 30.

As a result, the ultrasound diagnosis apparatus 1 according to the first embodiment is able to display a color Doppler image in which almost no clutter component is contained, from among the color Doppler images generated by using the optimal color gain. In other words, the ultrasound diagnosis apparatus 1 is able to display the color Doppler image that does not contain noises or clutter components and in which the bloodstream information is accurately displayed.

Further, according to the first embodiment, the quantifying unit 175 calculates, as the quantitative information that quantitatively indicates the bloodstream information included in the color Doppler image selected by the image selecting unit 174, the number of color pixels, the color pixel ratio, a sum of the power values, and/or the like and displays the calculated quantitative information.

As a result, the ultrasound diagnosis apparatus 1 according to the first embodiment is able to calculate the quantitative information from the color Doppler image that does not contain noises or clutter components and in which the bloodstream information is accurately displayed. Thus, the ultrasound diagnosis apparatus 1 is able to provide the operator with the quantitative information having a high reliability.

The various types of processes performed by the ultrasound diagnosis apparatus 1 according to the first embodiment are not limited to the examples described above. In the following sections, modification examples of the various types of processes performed by the ultrasound diagnosis apparatus 1 according to the first embodiment will be explained.

In the first embodiment, the example is explained in which the ultrasound diagnosis apparatus 1 selects an optimal color gain, and subsequently selects an optimal color Doppler image, before further calculating the quantitative information based on the optimal color Doppler image. It is however, acceptable if the ultrasound diagnosis apparatus 1 performs only the process of selecting the optimal color gain. In other words, the ultrasound diagnosis apparatus 1 does not necessarily have to include the image selecting unit 174 and the quantifying unit 175 shown in FIG. 4. Even with this configuration, the ultrasound diagnosis apparatus 1 is able to select an optimal color gain. Thus, the ultrasound diagnosis apparatus 1 is able to generate a color Doppler image in which almost no noise is contained. As another example, the ultrasound diagnosis apparatus 1 may perform the process of selecting the optimal color gain and the process of selecting the optimal color Doppler image, without performing the process of calculating the quantitative information. As yet another example, the ultrasound diagnosis apparatus 1 may perform the process of selecting the optimal color gain and the process of calculating the quantitative information, without performing the process of selecting the optimal color Doppler image.

Further, in the first embodiment, the example is explained in which the index calculating unit 172 calculates the color pixel ratios from the plurality of color Doppler images generated by using the mutually-different color gains as explained with reference to FIG. 5. However, the index calculating unit 172 may count the number of color pixels, instead of calculating the color pixel ratios. In that situation, the gain selecting unit 173 extracts the number of color pixels of the two color Doppler images corresponding to two adjacent color gains, from among the numbers of color pixels counted by the index calculating unit 172 and selects the smallest value of the group made up of the color gains by which the change amount between the two extracted numbers of color pixels is equal to or larger than the change-amount threshold value, as an optimal color gain.

Also, in the first embodiment, the example is explained in which the index calculating unit 172 calculates the color pixel ratio in the predetermined region of interest, from each of the plurality of color Doppler images generated by using the mutually-different color gains. However, the index calculating unit 172 may calculate the ratio between the number of pixels in the entire color Doppler image and the number of color pixels in the entire color Doppler image, as the color pixel ratio.

Further, in the first embodiment, the example is explained in which, when selecting an optimal color gain, the Doppler processing unit 132 stores the one piece of reflected-wave data input from the ultrasound receiving unit 120 into the storage unit 180 and calculates the bloodstream information for each of the color gains, by performing the gain adjusting process on the one piece of reflected-wave data by using the plurality of mutually-different color gains. However, when selecting the optimal color gain, the controlling unit 170 may control the ultrasound transmitting unit 110 so as to generate a plurality of pieces of reflected-wave data. In that situation, the Doppler processing unit 132 calculates the bloodstream information for each of the color gains, by changing the color gain every time a piece of reflected-wave data is input from the ultrasound receiving unit 120.

Further, in the first embodiment, the example is explained in which, as described with reference to FIGS. 5, 6, and 10, the gain selecting unit 173 sequentially extracts two adjacent color gains starting from the color gain minimum value and judges whether the change amount between the color pixel ratios is equal to or larger than the change-amount threshold value. In other words, in this example, the gain selecting unit 173 judges the change amounts in the color pixel ratios in the direction from the smaller color gains to the larger color gains. However, it is acceptable for the gain selecting unit 173 to judge the change amounts in the color pixel ratios in the direction from the larger color gains to the smaller color gains.

Further, in the first embodiment, the example is explained in which, as described with reference to FIGS. 5, 6, and 10, the gain selecting unit 173 selects the optimal color gain at the point in time when the change amount between the color pixel ratios is equal to or larger than the change-amount threshold value. However, the gain selecting unit 173 may calculate a change amount between the color pixel ratios corresponding to two adjacent color gains for each of all the sets made up of two adjacent color gains and may judge the change amounts between the color pixel ratios, starting from the color gain by which the change amount between the color pixel ratios is largest, toward the smaller color gains.

For example, let us assume that the various types of data stored in the pixel ratio storage unit 182 are in the state shown in FIG. 5. In that situation, the gain selecting unit 173 calculates the change amount between the color pixel ratios for each of all the sets made up of two adjacent color gains. In the example shown in FIG. 5, the change amount between the color pixel ratios corresponding to the color gains “59” and “60” is largest. In this situation, the gain selecting unit 173 judges the change amounts between the color pixel ratios starting from the color gain “59” toward the smaller color gains. More specifically, the gain selecting unit 173 subsequently extracts the two adjacent color gains “58” and “59” and judges the change amount between the color pixel ratios. If the change amount is smaller than the change-amount threshold value, the gain selecting unit 173 subsequently extracts the two adjacent color gains “57” and “58” and judges the change amount between the color pixel ratios.

By performing the judging process in this order, the gain selecting unit 173 is able to reduce the processing amount in the process of comparing the change amounts with the change-amount threshold value. As a result, it is possible to reduce the processing load. More specifically, when the color gain is gradually increased as in the example shown in FIG. 6, when the color gain exceeds the optimal color gain, the color pixel ratio of the color Doppler image radically increases at first and gradually increases later. In other words, the optimal color gain is present in the vicinity of the color gain by which the change amount between the color pixel ratios is largest. For this reason, by judging the change amounts between the color pixel ratios starting from the color gain by which the change amount between the color pixel ratios is largest, toward the smaller color gains, the gain selecting unit 173 is able to identify the optimal color gain by performing a small amount of judging process.

As additional information, if it has been learned that, for example, the optimal color gain is the color gain obtained by subtracting a predetermined value from the color gain by which the change amount between the color pixel ratios is largest, the gain selecting unit 173 may select the color gain that is smaller, by the predetermined value, than the color gain by which the change amount between the color pixel ratios is largest, as an optimal color gain.

In the first embodiment, the example is explained in which, when selecting the optimal color gain, the Doppler processing unit 132 calculates the plurality of pieces of bloodstream information, by using the plurality of color gains that are different from each other by the predetermined difference. For example, in FIG. 5, the Doppler processing unit 132 uses the color gains that are different from each other by “1”. However, the Doppler processing unit 132 may calculate a plurality of pieces of bloodstream information by using color gains between which the differences are not a constant value. For example, the Doppler processing unit 132 may calculate the bloodstream information by increasing the color gain by “2” from the color gain “35”, increasing the color gain by “1” after the color gain reaches “50”, and increasing the color gain by “2” after the color gain reaches “63”. In that situation, the gain selecting unit 173 does not compare the change amount in the color pixel ratios between two adjacent color gains with the change-amount threshold value, but compares the change amount in the color pixel ratios with respect to the change amount in the color gains, with a change-amount threshold value. For example, the gain selecting unit 173 compares a value obtained by dividing the change amount in the color pixel ratios between two adjacent color gains by the change amount in the color gains, with the change-amount threshold value.

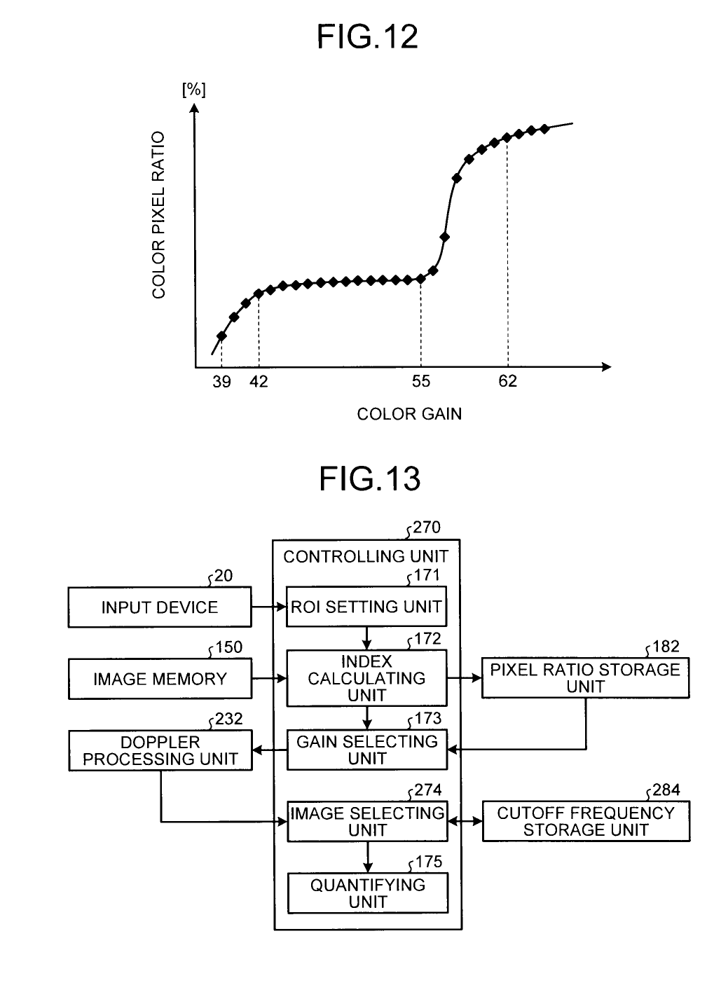

Further, in the first embodiment, the example is explained in which the gain selecting unit 173 extracts two adjacent color gains, starting from the color gain minimum value and judges whether the change amount between the color pixel ratios is equal to or larger than the change-amount threshold value. However, the gain selecting unit 173 may calculate the color pixel ratios for a predetermined range of color gains and may judge whether the change amount between the calculated color pixel ratios is equal to or larger than the change-amount threshold value. This example will be more specifically explained, with reference to FIG. 12. FIG. 12 is a chart of an exemplary relationship between color gains and color pixel ratios.

In the example shown in FIG. 12, the change amounts in the color pixel ratios of the color Doppler image are large between the color gain “39” and the color gain “42”. The change amounts in the color pixel ratios are small between the color gain “42” and the color gain “55”. The change amounts in the color pixel ratios are large between the color gain “55” and the color gain “62”. With such a relationship between the color gains and the color pixel ratios, if the color Doppler image is generated by using a color gain in the range from “39” to “41”, for example, the bloodstream information is not accurately displayed. If the color Doppler image is generated by using a color gain in the range from “42” to “55”, the bloodstream information is accurately displayed. If the color Doppler image is generated by using a color gain equal to or larger than “56”, noises are displayed in the whole area. As explained above, the optimal color gain is the largest value among the color gains by which almost no noise is displayed in the color Doppler image. Thus, in the example in FIG. 12, the optimal color gain is “55”.

Incidentally, in the example in FIG. 12, when the gain selecting unit 173 sequentially extracts two adjacent color gains, starting from the color gain minimum value, so as to calculate the change amount between the color pixel ratios, there is a possibility that the gain selecting unit 173 may determine that the change amount between the color pixel ratios corresponding to the color gains “39” and “40” is equal to or larger than the change-amount threshold value. To cope with this situation, the gain selecting unit 173 may extract two adjacent color gains with respect to, for example, a range of color gains from “45” to “60” and may judge whether the change amount between the color pixel ratios is equal to or larger than the change-amount threshold value. The range of color gains used in this situation is stored in the storage unit 180 in advance. When performing the process to select the optimal color gain, the gain selecting unit 173 obtains the range of color gains from the storage unit 180. As for the range of color gains, the storage unit 180 may store therein, for example, mutually-different values for different image-taking target sites.

As explained above, it is considered that the optimal color gain is present in the vicinity of the color gain by which the change amount between the color pixel ratios is largest. Accordingly, even if the relationship between color gains and color pixel ratios is as shown in the example in FIG. 12, the gain selecting unit 173 is able to select the optimal color gain by judging the change amounts between the color pixel ratios, starting from the color gain by which the change amount between the color pixel ratios is largest, toward the smaller color gains.

### Second Embodiment

In the first embodiment above, the example is explained in which the image selecting unit 174 selects the color Doppler image of which the number of color pixels is either the smallest value or the largest value as the optimal color Doppler image, from among the plurality of color Doppler images generated for mutually the same image taking position. In a second embodiment, an example will be explained in which an optimal color Doppler image is selected without using information such as the numbers of color pixels calculated from the color Doppler images.

First, a controlling unit according to the second embodiment will be explained, with reference to FIG. 13. FIG. 13 is a block diagram of an exemplary configuration of a controlling unit 270 according to the second embodiment. In the following sections, some of the processing units that have the same functions as those of the processing units shown in FIG. 4 will be referred to by using the same reference characters, and the detailed explanation thereof will be omitted.

Also, the configuration of an ultrasound diagnosis apparatus according to the second embodiment is similar to the exemplary configuration shown in FIG. 1, except that, as shown in FIG. 13, the ultrasound diagnosis apparatus according to the second embodiment includes a Doppler processing unit 232 in place of the Doppler processing unit 132 shown in FIG. 1. Also, the storage unit 180 according to the second embodiment includes a cutoff frequency storage unit 284. Further, as shown in FIG. 13, the controlling unit 270 according to the second embodiment includes an image selecting unit 274.

The Doppler processing unit 232 includes a wall filter of which the cutoff frequency is variable. More specifically, the Doppler processing unit 232 sets such a filter characteristic into the wall filter that, based on the reception signal received by the ultrasound probe 10, optimally separates a bloodstream signal and a clutter signal contained in the reception signal (see U.S. Pat. No. 6,224,557). In other words, the Doppler processing unit 232 sets a high cutoff frequency if the reception signal contains a large number of clutter components, whereas the Doppler processing unit 232 sets a low cutoff frequency if the reception signal contains a small number of clutter components. The wall filter included in the Doppler processing unit 232 is, for example, a Moving Target Indicator (MTI), a clutter eliminating filter, or the like.

From among the plurality of color Doppler images that are generated for mutually the same image taking position and are stored in the image memory 150, the image selecting unit 274 selects a color Doppler image of which the cutoff frequency in the wall filter used in the generating process performed by the image generating unit 140 is lowest, as an optimal color Doppler image.

More specifically, every time the image generating unit 140 generates a color Doppler image, the image selecting unit 274 stores the cutoff frequency set by the Doppler processing unit 232 into the cutoff frequency storage unit 284. In this situation, the image selecting unit 274 stores image identification information identifying the color Doppler image generated by the image generating unit 140 and the cutoff frequency, which are kept in correspondence with each other, into the cutoff frequency storage unit 284. In the same manner as in the first embodiment, the image generating unit 140 generates a color Doppler image from the bloodstream information on which the gain adjusting process is performed by using the optimal color gain.

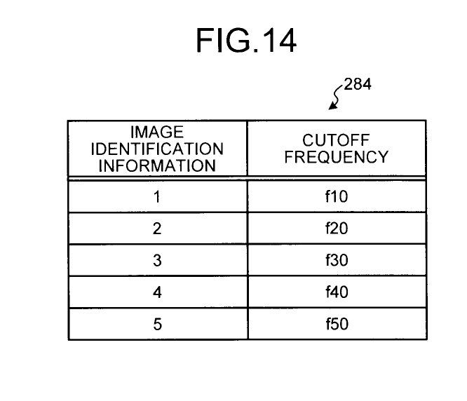

Next, the cutoff frequency storage unit 284 will be explained, with reference to FIG. 14. FIG. 14 is a drawing of an example of the cutoff frequency storage unit 284. As shown in FIG. 14, the cutoff frequency storage unit 284 includes items such as “image identification information” and “cutoff frequency”.

The item “image identification information” denotes information for identifying each of the color Doppler images. The item “cutoff frequency” denotes the cutoff frequency set to the wall filter included in the Doppler processing unit 232, when the color Doppler image identified by the corresponding piece of image identification information is generated.

For example, the cutoff frequency storage unit 284 shown in FIG. 14 indicates that the color Doppler image identified by the image identification information “1” is generated, while a cutoff frequency “f10” is set to the wall filter included in the Doppler processing unit 232. As another example, the cutoff frequency storage unit 284 shown in FIG. 14 indicates that the color Doppler image identified by the image identification information “2” is generated while a cutoff frequency “f20” is set to the wall filter included in the Doppler processing unit 232.

The image selecting unit 274 stores the various types of data into the cutoff frequency storage unit 284 in this manner and, when having received a request from the operator indicating that an optimal color Doppler image should be displayed, the image selecting unit 274 selects the color Doppler image of which the cutoff frequency stored in the cutoff frequency storage unit 284 is the smallest value, as an optimal color Doppler image. For example, in the example in FIG. 14, let us assume that the cutoff frequency “f10” is the smallest value among the cutoff frequencies “f10”, “f20”, “f30”, “f40”, and “f50”. In this situation, the image selecting unit 274 selects the color Doppler image identified by the image identification information “1” corresponding to the cutoff frequency “f10”, as an optimal color Doppler image.

In the following sections, the reason why it is possible to select the optimal color Doppler image through the process performed by the image selecting unit 274 described above will be explained. As explained in the first embodiment, in the plurality of color Doppler images generated by the image generating unit 140, almost no noise is supposed to be displayed, and also, the bloodstream information and the like are supposed to be accurately displayed, but there is a possibility that clutter components may be contained. In this situation, the Doppler processing unit 232 is configured so as to set the cutoff frequencies in such a manner that the more clutter components are contained in the color Doppler image, the higher is the cutoff frequency and that the fewer clutter components are contained in the color Doppler image, the lower is the cutoff frequency. In other words, the lower the cutoff frequency is set by the Doppler processing unit 232, the fewer clutter components are contained in the color Doppler image.

For this reason, from among the plurality of color Doppler images generated by the image generating unit 140 for mutually the same image taking position, the image selecting unit 274 according to the second embodiment selects the color Doppler image of which the cutoff frequency used during the image generating process thereof is the lowest value, as an optimal color Doppler image.

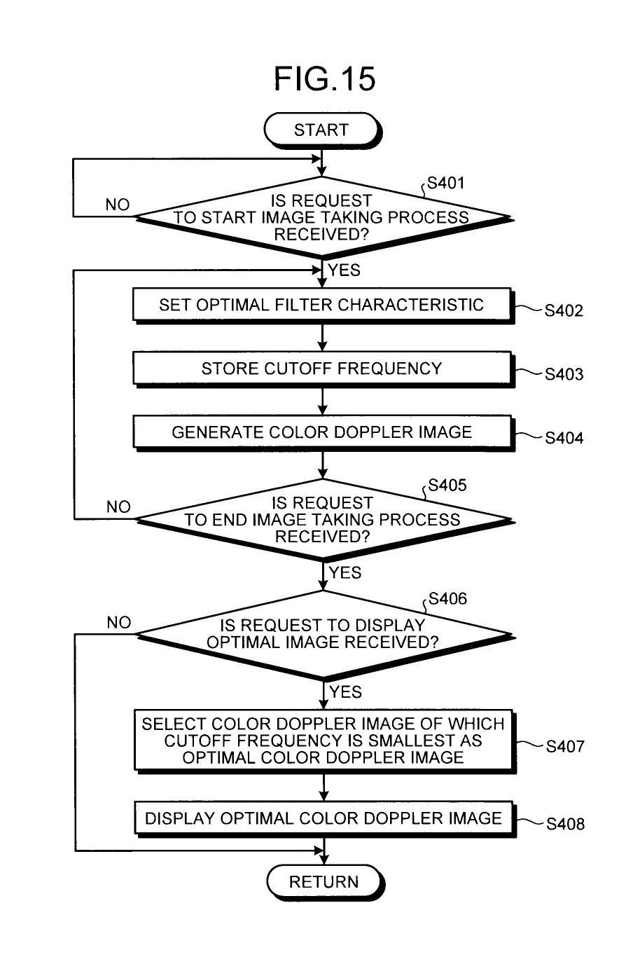

Next, a processing procedure performed by the ultrasound diagnosis apparatus according to the second embodiment will be explained, with reference to FIG. 15. FIG. 15 is a flowchart of the processing procedure performed by the ultrasound diagnosis apparatus according to the second embodiment. In the following sections, it is assumed that the optimal gain has been set by the gain selecting unit 173.

As shown in FIG. 15, the ultrasound diagnosis apparatus according to the second embodiment judges whether a request to start an image taking process is received from the operator (step S401). If no request to start an image taking process is received (step S401: No), the ultrasound diagnosis apparatus goes into a stand-by state.

On the contrary, if a request to start an image taking process is received (step S401: Yes), the Doppler processing unit 232 estimates an optimal filter characteristic and sets the estimated filter characteristic into the wall filter (step S402). In this situation, the image selecting unit 274 stores the cutoff frequency set by the Doppler processing unit 232 into the cutoff frequency storage unit 284 (step S403).

By using the wall filter in which the optimal filter characteristic has been set, the Doppler processing unit 232 calculates bloodstream information from the reflected-wave data input from the ultrasound receiving unit 120. Further, the image generating unit 140 generates a color Doppler image from the bloodstream information calculated by the Doppler processing unit 232 (step S404). In this situation, the image selecting unit 274 stores the image identification information of the color Doppler image generated by the image generating unit 140 and the cutoff frequency, which are kept in correspondence with each other, into the cutoff frequency storage unit 284.

Further, the controlling unit 270 judges whether a request to end the image taking process is received from the operator (step S405). If no request to end the image taking process is received (step S405: No), the process returns to step S402.

On the contrary, if a request to end the image taking process is received (step S405: Yes), the image selecting unit 274 judges whether a request indicating that an optimal color Doppler image should be displayed is received from the operator (step S406). In this situation, if no request indicating that an optimal color Doppler image should be displayed is received from the operator (step S406: No), the image selecting unit 274 ends the process.

On the contrary, if a request indicating that an optimal color Doppler image should be displayed is received from the operator (step S406: Yes), the image selecting unit 274 selects the color Doppler image of which the cutoff frequency stored in the cutoff frequency storage unit 284 is the smallest value, as an optimal color Doppler image (step S407). After that, the image selecting unit 274 displays the optimal color Doppler image on the monitor 30 (step S408).

As explained above, according to the second embodiment, the Doppler processing unit 232 sets the filter characteristic of the wall filter based on the reception signal received by the ultrasound probe 10 and eliminates the predetermined frequency band from the reception signal. After that, the image generating unit 140 controlled by the controlling unit 170 so as to perform the image generating process by using the optimal color gain generates the plurality of color Doppler images for mutually the same image taking position from the bloodstream information generated by the Doppler processing unit 232 and stores the generated plurality of color Doppler images into the image memory 150. After that, from among the plurality of color Doppler images that are generated for mutually the same image taking position and are stored in the image memory 150, the image selecting unit 274 selects the color Doppler image of which the cutoff frequency of the wall filter used in the generating process performed by the image generating unit 140 is the lowest value, as the optimal color Doppler image, and displays the selected color Doppler image on the monitor 30.

With these arrangements, the ultrasound diagnosis apparatus according to the second embodiment is able to select the optimal color Doppler image without calculating the numbers of color pixels or the color pixel ratios of the color Doppler images. Thus, it is possible to prevent the processing load from increasing and to display a color Doppler image in which almost no clutter component is contained. In other words, the ultrasound diagnosis apparatus according to the second embodiment is able to prevent the processing load from increasing and to display the color Doppler image that does not contain noises or clutter components and in which the bloodstream information is accurately displayed.

### Third Embodiment

In the first embodiment, the example is explained in which the optimal color gain is selecting by transmitting the ultrasound wave to the patient P and generating the color Doppler images by using the reflected-wave signal from the patient P. In a third embodiment, an example will be explained in which an optimal color gain is selected without transmitting an ultrasound wave to the patient P.

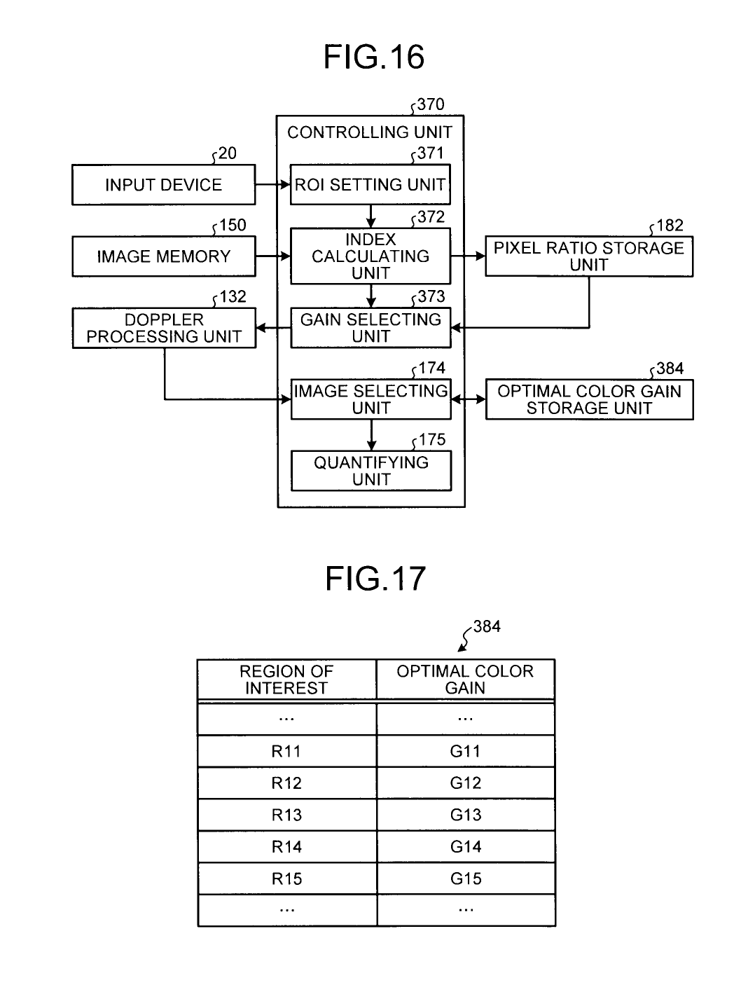

First, a controlling unit according to the third embodiment will be explained, with reference to FIG. 16. FIG. 16 is a block diagram of an exemplary configuration of the controlling unit according to the third embodiment. In the following sections, some of the processing units that have the same functions as those of the processing units shown in FIG. 4 will be referred to by using the same reference characters, and the detailed explanation thereof will be omitted.

Also, the configuration of an ultrasound diagnosis apparatus according to the third embodiment is similar to the exemplary configuration shown in FIG. 1, except that, as shown in FIG. 16, the storage unit 180 according to the third embodiment includes an optimal color gain storage unit 384. Further, as shown in FIG. 16, a controlling unit 370 according to the third embodiment includes an ROI setting unit 371, an index calculating unit 372, and a gain selecting unit 373.

When having received a request to start an image taking process from the operator, the ROI setting unit 371 shifts the position in which a region of interest is set at regular time intervals, while the process performed by the ultrasound transmitting unit 110 is being stopped. In other words, the ROI setting unit 371 shifts the setting position of the region of interest, after the transmitting function of the ultrasound diagnosis apparatus is stopped, and the receiving function thereof is activated.

As a result, the ultrasound receiving unit 120, the Doppler processing unit 132, and the image generating unit 140 do not receive the reflected-wave signal from the patient P, but receive a noise signal and the like occurring in the surrounding of the ultrasound diagnosis apparatus and generate a color Doppler image from the noise signal and the like.

Further, like in the first embodiment, the Doppler processing unit 132 according to the third embodiment calculates bloodstream information for each of the color gains, by performing the gain adjusting process on the data input from the ultrasound receiving unit 120, by using the plurality of color gains that are different from each other by the predetermined difference. Further, the image generating unit 140 generates color Doppler images from the bloodstream information calculated by the Doppler processing unit 132.

Further, the index calculating unit 372 counts the number of color pixels for each of the color Doppler images generated by the image generating unit 140. In this situation, the index calculating unit 372 counts the number of color pixels in a region of interest set by the ROI setting unit 371. Further, the index calculating unit 372 calculates a color pixel ratio that is the ratio between the counted number of color pixels and the total number of pixels in the region of interest.

Further, the gain selecting unit 373 according to the third embodiment selects an optimal color gain for each of the regions of interest, based on the color pixel ratios calculated by the index calculating unit 372. The relationship between the color pixel ratios calculated by the index calculating unit 372 and the color gains exhibits the same waveform as in the example in FIG. 6. Accordingly, the gain selecting unit 373 is able to select an optimal color gain by performing the same process as in the first embodiment.

In other words, the index calculating unit 372 and the gain selecting unit 373 according to the third embodiment select an optimal color gain for each of the regions of interest, by performing the same process as in the first embodiment for each of the regions of interest set by the ROI setting unit 371. After that, the gain selecting unit 373 stores the regions of interest and the optimal color gains, which are kept in correspondence with one another, into the optimal color gain storage unit 384.

Next, the optimal color gain storage unit 384 will be explained, with reference to FIG. 17. FIG. 17 is a drawing of an example of the optimal color gain storage unit 384. As shown in FIG. 17, the optimal color gain storage unit 384 includes items such as “region of interest” and “optimal color gain”.

The item “region of interest” denotes each of the regions of interest set by the ROI setting unit 371. The item “optimal color gain” denotes each of the optimal color gains selected by the gain selecting unit 373. For example, the optimal color gain storage unit 384 shown in FIG. 17 indicates that, when the region of interest is set to “R11”, the optimal color gain is “G11”. As another example, the optimal color gain storage unit 384 shown in FIG. 17 indicates that, when the region of interest is set to “R12”, the optimal color gain is “G12”.

After an optimal color gain is selected for each of the regions of interest in this manner, the ultrasound diagnosis apparatus according to the third embodiment generates a color Doppler image according to an instruction from the operator. In this situation, the Doppler processing unit 132 obtains the color gain corresponding to the region of interest set by the operator from the optimal color gain storage unit 384 and calculates bloodstream information by using the obtained color gain.

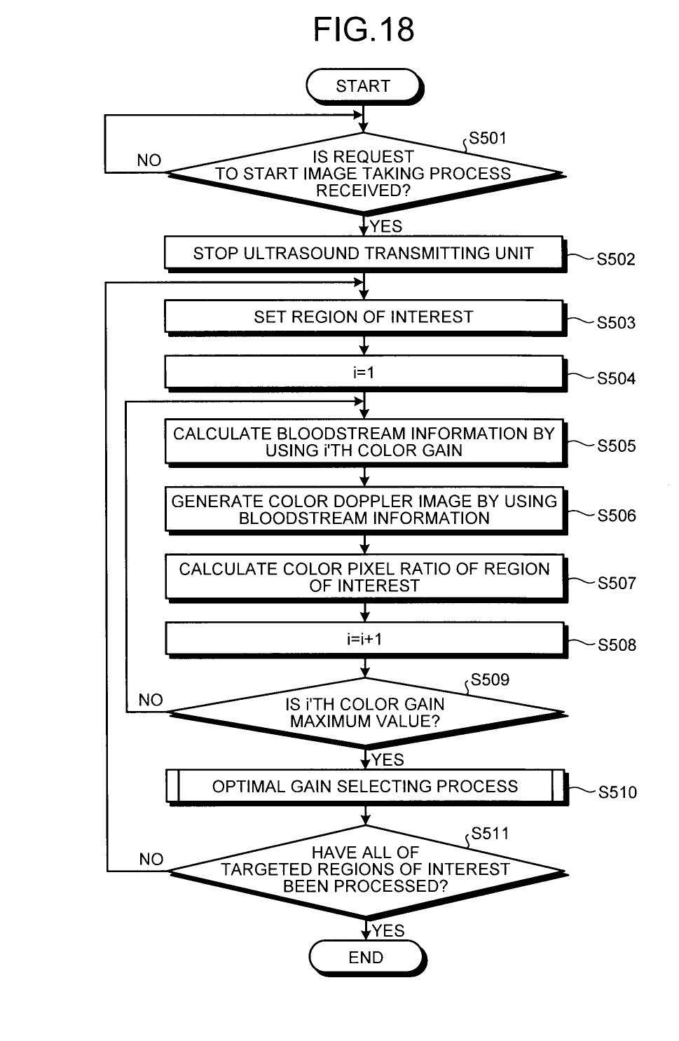

Next, a procedure in the optimal gain selecting process performed by the ultrasound diagnosis apparatus according to the third embodiment will be explained, with reference to FIG. 18. FIG. 18 is a flowchart of the procedure in the optimal gain selecting process performed by the ultrasound diagnosis apparatus according to the third embodiment.

As shown in FIG. 18, the ultrasound diagnosis apparatus according to the third embodiment judges whether a request to start an image taking process is received from the operator (step S501). If no request to start an image taking process is received (step S501: No), the ultrasound diagnosis apparatus goes into a stand-by state.

On the contrary, if a request to start an image taking process is received (step S501: Yes), the controlling unit 370 stops the process performed by the ultrasound transmitting unit 110 (step S502) and sets a region of interest (step S503). Subsequently, the ROI setting unit 371 sets the counter i to “1” (step S504).

After that, under the control of the controlling unit 370, the Doppler processing unit 132 calculates bloodstream information by performing a gain adjusting process on reflected-wave data input from the ultrasound receiving unit 120 by using an i'th color gain (step S505). Further, the image generating unit 140 generates a color Doppler image from the bloodstream information calculated by the Doppler processing unit 132 (step S506). After that, the index calculating unit 372 calculates a color pixel ratio of the region of interest set at step S503 within the color Doppler image generated by the image generating unit 140 (step S507).

Subsequently, the controlling unit 370 adds “1” to the counter i (step S508) and judges whether the i'th color gain is larger than the color gain maximum value of which the Doppler processing unit 132 is informed (step S509). In this situation, if the i'th color gain is equal to or smaller than the color gain maximum value (step S509: No), the process returns to step S505.

On the contrary, if the i'th color gain is larger than the color gain maximum value (step S509: Yes), the gain selecting unit 373 performs the optimal gain selecting process (step S510). The optimal gain selecting process performed by the gain selecting unit 373 is the same as the optimal gain selecting process shown in FIG. 10. In this situation, the gain selecting unit 373 stores information identifying the region of interest set at step S503 and the optimal color gain selected in the optimal gain selecting process, which are kept in correspondence with each other, into the optimal color gain storage unit 384.

After that, the controlling unit 370 judges whether the optimal gain selecting process has been performed for each of all the regions of interest that are the targets of the optimal gain selecting process (step S511). In this situation, if the optimal gain selecting process has not been performed on each of all the regions of interest (step S511: No), the process returns to step S503, so that the ROI setting unit 371 sets an unprocessed region of interest (step S503).

On the contrary, if the optimal gain selecting process has been performed on each of all the regions of interest (step S511: Yes), the process is ended. After that, to generate a color Doppler image, the ultrasound diagnosis apparatus is configured so that the Doppler processing unit 132 obtains the color gain corresponding to the region of interest set by the operator from the optimal color gain storage unit 384 and calculates bloodstream information by using the obtained color gain.

As explained above, according to the third embodiment, the ROI setting unit 371 shifts the region of interest at the regular time intervals, while the process performed by the ultrasound transmitting unit 110 is being stopped. Further, the image generating unit 140 generates the color Doppler image for each of the regions of interest shifted by the ROI setting unit 371. After that, the index calculating unit 372 counts the number of color pixels for each of the color Doppler images generated by the image generating unit 140. Subsequently, for each of the regions of interest shifted by the ROI setting unit 371, the gain selecting unit 373 extracts the numbers of color pixels of the two color Doppler images corresponding to two adjacent color gains, from among the numbers of color pixels counted by the index calculating unit 372, and further selects the smallest value of a group made up of the color gains by which the change amount between the two extracted numbers of color pixels is equal to or larger than the change-amount threshold value, as an optimal color gain.

With these arrangements, the ultrasound diagnosis apparatus according to the third embodiment is able to set the optimal color gain. In particular, the ultrasound diagnosis apparatus according to the third embodiment is able to set the optimal color gain by performing only the receiving process, without performing the transmitting process. Thus, it is possible to set a color gain by which the system noises occurring in the internal circuits and the like of the ultrasound diagnosis apparatus are not displayed in the color Doppler image.

Further, the ultrasound diagnosis apparatus according to the third embodiment sets an optimal color gain for each of the regions of interest. Thus, the ultrasound diagnosis apparatus is able to generate, for each of the regions of interest, a color Doppler image that has a low possibility of containing noises and in which the bloodstream information is accurately displayed. For example, when the ROI setting unit 371 shifts the region of interest in each of the depth directions from the ultrasound probe 10 toward the interior of the body of the patient, the ultrasound diagnosis apparatus according to the third embodiment is able to set an optimal color gain for each of the depth directions.

In the third embodiment described above, the example is explained in which the color gain is estimated each of the regions of interest while an ultrasound wave transmitting process performed by the ultrasound probe is being stopped. However, other examples are also acceptable in which the ultrasound diagnosis apparatus according to the third embodiment does not shift the region of interest, and estimates an color gain to all the regions of interest while an ultrasound wave transmitting process performed by the ultrasound probe is being stopped. In other words, the ultrasound diagnosis apparatus may set a typical optimal color gain to be applied to the whole color image domain. The ultrasound diagnosis apparatus may select the average value of the color gains that are selected each of the regions of interest or may select the minimum or the maximum among the color gains as the setting method of the typical color gain. Furthermore, the number of regions of interest may be plurality or one.

### Fourth Embodiment

In the first embodiment described above, the example is explained in which the index calculating unit 172 calculates the color pixel ratio as an index value indicating the distribution of the color Doppler image. In a fourth embodiment, an example will be explained in which information other than the color pixel ratio is calculated as an index value indicating the distribution of the color Doppler image.

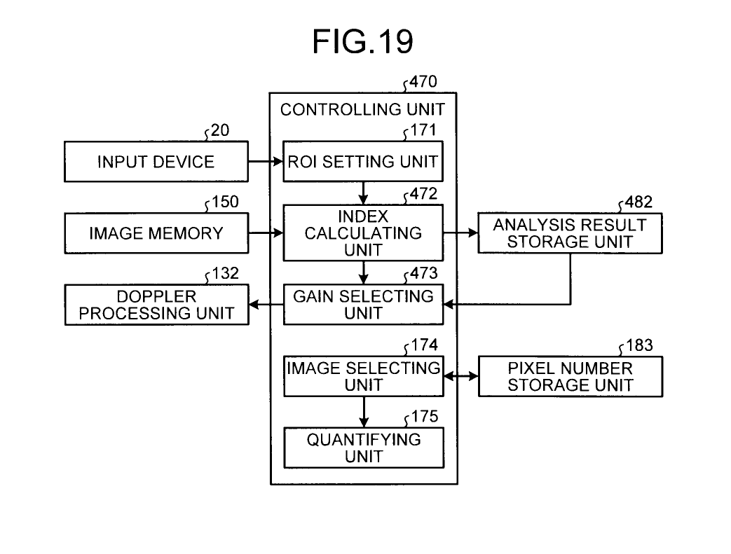

First, a controlling unit according to the fourth embodiment will be explained, with reference to FIG. 19. FIG. 19 is a block diagram of an exemplary configuration of a controlling unit 470 according to the fourth embodiment. In the following sections, some of the processing units that have the same functions as those of the processing units shown in FIG. 4 will be referred to by using the same reference characters, and the detailed explanation thereof will be omitted.

Also, the configuration of an ultrasound diagnosis apparatus according to the fourth embodiment is similar to the exemplary configuration shown in FIG. 1, except that, as shown in FIG. 19, the storage unit 180 according to the fourth embodiment includes an analysis result storage unit 482. Also, as shown in FIG. 19, the controlling unit 470 according to the fourth embodiment includes an index calculating unit 472 and a gain selecting unit 473.

By analyzing, for each of the color Doppler images generated by the image generating unit 140, the color pixels allocated in the color Doppler image, the index calculating unit 472 calculates a distribution formation of the color pixels contained in the color Doppler image as an index value.

More specifically, when the image generating unit 140 has generated color Doppler images respectively corresponding to the color gains, the index calculating unit 472 sequentially obtains the color Doppler images from the image memory 150 and calculates a distribution formation of the color pixels contained in a region of interest in each of the color Doppler images. Further, the index calculating unit 472 stores the color gains used in the generating process of the color Doppler images and pieces of distribution formation information each indicating the distribution formation of the color pixels in a corresponding one of the color Doppler images, which are kept in correspondence with one another, into the analysis result storage unit 482.

The gain selecting unit 473 selects a display-purpose color gain, by comparing the distribution formations of the color pixels calculated by the index calculating unit 472 as index values. More specifically, the gain selecting unit 473 extracts two pieces of distribution formation information corresponding to two adjacent color gains from among the pieces of distribution formation information stored in the analysis result storage unit 482 and, when a change amount between the two extracted pieces of distribution formation information is equal to or larger than a change-amount threshold value, the gain selecting unit 473 selects the smaller of the two color gains as an optimal color gain.

A specific example will be explained. Like the gain selecting unit 173 according to the first embodiment, the gain selecting unit 473 sequentially obtains two pieces of distribution formation information corresponding to two adjacent color gains, starting from the color gain minimum value. Further, the gain selecting unit 473 calculates a degree of similarity between the two distribution formations by, for example, performing a pattern analysis on the two distribution formations indicated by the pieces of distribution formation information. Further, when the degree of similarity is equal to or larger than a change-amount threshold value, the gain selecting unit 473 selects the smaller of the two color gains, as an optimal color gain.

Next, the reason why it is possible to select the optimal color gain through the process performed by the gain selecting unit 473 will be explained. As explained in the first embodiment, ultrasound diagnosis apparatuses have a characteristic where, when the color gain is gradually increased, the amount of noises contained in the color Doppler image radically increases at a predetermined color gain serving as a border line. In other words, generally speaking, the color pixel ratio is low when the color gain is small, whereas the color pixel ratio is high when the color gain is large. For this reason, when the color gain is gradually increased, the distribution formations of the color pixels in the color Doppler images generated by using mutually-different color gains do not have a large difference therebetween until the color gain reaches the predetermined value. It is considered that, however, when the color gain has reached the predetermined value so that the amount of noises radically increases, the distribution formation of the color pixels in the color Doppler image changes significantly.

For example, let us assume that the relationship between color gains and color pixel ratios is as shown in the example in FIG. 6. In that situation, while the color gain is in the range from “38” to “54”, because the color pixel ratio does not change significantly, each of all the color Doppler images looks like the color Doppler image shown in FIG. 2. In contrast, when the color gain is “55” or larger, because the color pixel ratio changes significantly, as the color gain is increased, the color Doppler image radically changes from the one shown in FIG. 2 to the one shown in FIG. 3. When the color Doppler image shown in FIG. 2 is compared with the color Doppler image shown in FIG. 3, it is observed that there is a large difference between the distribution formations of the color pixels. For this reason, by selecting the color gain “55”, the gain selecting unit 473 is able to select the largest value from among the color gains by which almost no noise is displayed in the color Doppler image.

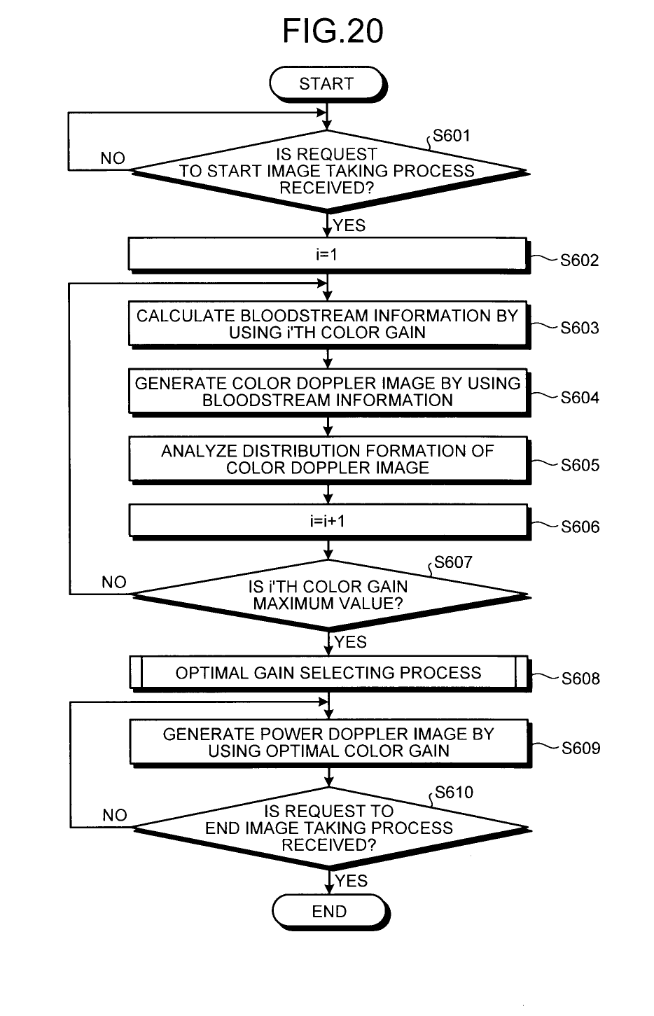

Next, a procedure in a process performed by the ultrasound diagnosis apparatus according to the fourth embodiment will be explained, with reference to FIG. 20. FIG. 20 is a flowchart of a processing procedure performed by the ultrasound diagnosis apparatus according to the fourth embodiment. Of the processing procedure shown in FIG. 20, the processes performed at steps S605 and S608 are different from the processing procedure shown in FIG. 10. Thus, the processes performed at steps S605 and S608 will be primarily explained in the following sections.

As shown in FIG. 20, the image generating unit 140 according to the fourth embodiment generates a color Doppler image from the bloodstream information calculated by the Doppler processing unit 132 (step S604). Further, the index calculating unit 472 included in the controlling unit 470 obtains a distribution formation of the color pixels contained in the color Doppler image, by analyzing the color Doppler image generated by the image generating unit 140 (step S605). Further, the index calculating unit 472 stores distribution formation information indicating the distribution formation and the color gain, which are kept in correspondence with each other, into the analysis result storage unit 482.

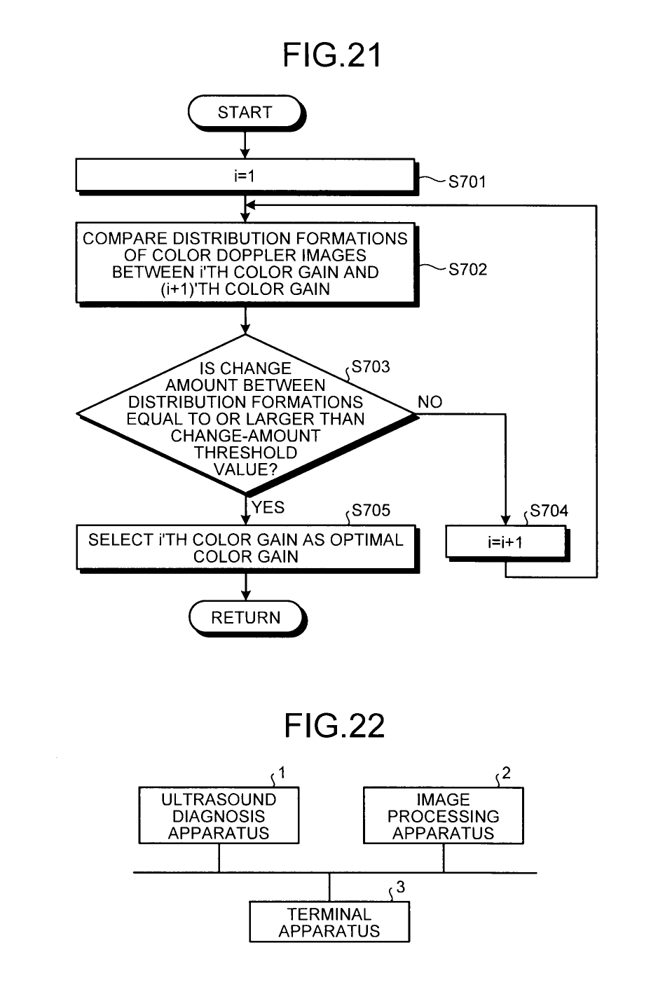

After that, the controlling unit 470 adds “1” to the counter i (step S606) and judges whether the i'th color gain is larger than the color gain maximum value of which the Doppler processing unit 132 is informed (step S607). Subsequently, the gain selecting unit 473 performs an optimal gain selecting unit (step S608). The optimal gain selecting process performed by the gain selecting unit 473 will be explained in detail later, with reference to FIG. 21.

After that, the Doppler processing unit 132, the image generating unit 140, and the like generate a color Doppler image by using the optimal color gain selected by the gain selecting unit 473 (step S609).

Next, a procedure in the optimal gain selecting process at step S608 in FIG. 20 will be explained, with reference to FIG. 21. FIG. 21 is a flowchart of the procedure in the optimal gain selecting process performed by the gain selecting unit 473 according to the fourth embodiment.

As shown in FIG. 21, the gain selecting unit 473 sets the counter i to “1” (step S701). Further, the gain selecting unit 473 obtains the distribution formation information corresponding to the i'th color gain and the distribution formation information corresponding to the (i+1)'th color gain from the analysis result storage unit 482 and compares the two obtained pieces of distribution formation information with each other (step S702). For example, the gain selecting unit 473 calculates a degree of similarity between the two distribution formations as a change amount between the distribution formations.

Further, the gain selecting unit 473 judges whether the change amount between the distribution formations is equal to or larger than a change-amount threshold value (step S703). In this situation, if the change amount between the distribution formations is smaller than the change-amount threshold value (step S703: No), the gain selecting unit 473 adds “1” to the counter i (step S704), and the process returns to step S702.

On the contrary, if the change amount between the distribution formations is equal to or larger than the change-amount threshold value (step S703: Yes), the gain selecting unit 473 selects the i'th color gain as an optimal color gain (step S705).

As explained above, according to the fourth embodiment, the index calculating unit 472 calculates the index value by analyzing the distribution formation of the color pixels allocated in each of the color Doppler images. The gain selecting unit 473 selects the optimal color gain by comparing the index values resulting from the analysis performed with respect to the plurality of color gains. With these arrangements, the ultrasound diagnosis apparatus according to the fourth embodiment is able to set the optimal color gain.

As another method according to the fourth embodiment by which a pattern analysis is used, it is also acceptable to calculate index values corresponding to the formations by analyzing the distribution formations. In that situation, control is exercised so that, if the distribution formation is determined to be close to a formation derived from noises, the index calculating unit 472 assigns a large index value. More specifically, distribution formations are expressed by using index values by, for example, storing a score table into the index calculating unit 472 in advance and reading the score table, the score table keeping the distribution formations of color pixels and the index values in correspondence with one another. For example, in the store table, a small index value is assigned to a formation in which the color pixels are distributed on a straight line; a large index value is assigned to a formation in which the color pixels are distributed in circular shapes; and an even larger index value is assigned to a formation in which the color pixels are distributed in the entire analyzed area without any exterior shape. With these arrangements, it is possible to assign a large index value to the color pixels derived from noises, by utilizing a phenomenon where the color pixels derived from noises appear randomly.

The first through the fourth embodiments are explained above by using the bloodstream information as an example of the information displayed in the color Doppler images; however, the ultrasound diagnosis apparatuses according to the first through the fourth embodiments are also applicable to an example in which information other than the bloodstream information is displayed in a color Doppler image. For example, the ultrasound diagnosis apparatuses according to the first through the fourth embodiments are also applicable to an example in which movement information of tissues are displayed in a color Doppler image by using a tissue Doppler method.

Further, in the first through the fourth embodiments, the example is described in which the Doppler processing unit 132 or 232 performs the gain adjusting process on the data input from the ultrasound receiving unit 120 by using the color gain; however, another example is acceptable in which the preamplifier 121 performs a gain adjusting process on the reception signal received by the ultrasound probe 10, by using a color gain. In that situation, the preamplifier 121 according to any of the first through the fourth embodiments uses the optimal color gain selected by the gain selecting unit 173 or the like.

Further, in the first, the second, and the fourth embodiments described above, the examples are explained in which the number of color pixels within the region of interest set in the color Doppler image is counted and in which the distribution formation of the color pixels within the region of interest is analyzed; however, other examples are also acceptable in which the index calculating unit 172 counts the number of color pixels of the entire color Doppler image and in which the index calculating unit 472 analyzes the distribution formation of the color pixels in the entire color Doppler image.

Further, another example is acceptable in which, like the index calculating unit 472 according to the fourth embodiment, the index calculating unit 372 according to the third embodiment analyzes the distribution formation of the color pixels in the color Doppler image, instead of counting the number of color pixels. Further, like the gain selecting unit 473 according to the fourth embodiment, the gain selecting unit 373 may select an optimal color gain based on the changes in the distribution formations.

The processes performed by any of the ultrasound diagnosis apparatuses according to the first through the fourth embodiments may be performed by an ultrasound diagnosis apparatus and an image processing apparatus. This feature will be explained, with reference to FIG. 22. FIG. 22 is a diagram of an exemplary configuration of an image processing system. The image processing system illustrated in FIG. 22 includes an ultrasound diagnosis apparatus 1, an image processing apparatus 2 such as a workstation or the like, and a terminal apparatus 3.

The ultrasound diagnosis apparatus 1 and the image processing apparatus 2 perform the processes performed by any of the ultrasound diagnosis apparatuses described in the first through the fourth embodiments. For example, the ultrasound diagnosis apparatus 1 includes the ultrasound probe 10, the ultrasound transmitting unit 110, the ultrasound receiving unit 120, the B-mode processing unit 131, and the Doppler processing unit 132 that are shown in FIG. 1, whereas the image processing apparatus 2 includes the image generating unit 140, the image memory 150, the image synthesizing unit 160, the controlling unit 170, the storage unit 180, and the interface unit 190. With these arrangements, the ultrasound diagnosis apparatus 1 and the image processing apparatus 2 perform the processes performed by any of the ultrasound diagnosis apparatuses described in the first through the fourth embodiments. In another example, the ultrasound diagnosis apparatus 1 includes the ultrasound probe 10, the ultrasound transmitting unit 110, the ultrasound receiving unit 120 that are shown in FIG. 1, whereas the image processing apparatus 2 includes the B-mode processing unit 131, the Doppler processing unit 132, the image generating unit 140, the image memory 150, the image synthesizing unit 160, the controlling unit 170, the storage unit 180, and the interface unit 190. With these arrangements, the ultrasound diagnosis apparatus 1 and the image processing apparatus 2 perform the processes performed by any of the ultrasound diagnosis apparatuses described in the first through the fourth embodiments. In any of these examples, both the ultrasound diagnosis apparatus 1 and the image processing apparatus 2 each include a CPU and an interface unit. As explained here, the processes performed by any of the ultrasound diagnosis apparatuses described in the first through the fourth embodiments can be performed by the ultrasound diagnosis apparatus 1 and the image processing apparatus 2. In the example shown in FIG. 22, the terminal apparatus 3 is an information processing apparatus such as a personal computer (PC) used by a user such as a medical doctor. The terminal apparatus 3 obtains a color Doppler image or the like stored in the image processing apparatus 2 and exercises control so that the obtained color Doppler image or the like is displayed by a predetermined display unit.

As explained above, according to the first through the fourth embodiments, it is possible to set the optimal color gain.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a movement information obtaining unit configured to obtain movement information by adjusting a reception signal received by an ultrasound probe, by using a gain;
an image generating unit configured to generate a color image in which color pixels are allocated based on the movement information;
an index calculating unit configured to calculate an index value indicating a distribution of the color pixels, based on two or more of the color images generated by the image generating unit;
a gain selecting unit configured to select a display gain by comparing the index values calculated with respect to two or more of the gains; and
a controlling unit configured to control the image generating unit so as to generate a color image in which color pixels are allocated based on the display gain and the movement information obtained from the reception signal.

2. The ultrasound diagnosis apparatus according to claim 1, wherein
the index calculating unit calculates a quantity of the color pixels allocated in the color image as the index value, and
the gain selecting unit selects the display gain by comparing the index values calculated with respect to two or more of the gains.

3. The ultrasound diagnosis apparatus according to claim 1, wherein
the index calculating unit calculates the index value by analyzing a distribution formation of the color pixels allocated in the color image, and
the gain selecting unit selects the display gain by comparing the index values resulting from the analysis performed with respect to two or more of the gains.

4. The ultrasound diagnosis apparatus according to claim 1, wherein
the index calculating unit calculates a proportion of the color pixels that are present within an area in which the color pixels are allocated, as the index value, and
the gain selecting unit selects the display gain by comparing the index values calculated with respect to two or more of the gains.

5. The ultrasound diagnosis apparatus according to claim 1, further comprising: a region-of-interest setting unit configured to set a region of interest that is an area in which the color pixels are allocated within the color image, wherein
the gain selecting unit selects the display gain by comparing the index values in the region of interest.

6. The ultrasound diagnosis apparatus according to claim 1, further comprising: an image storage unit configured to store therein a plurality of color images that are generated by the image generating unit for a mutually same image taking position under the control of the controlling unit, wherein
the controlling unit includes: an image selecting unit configured to select, from among the plurality of color images that are generated for mutually the same image taking position and are stored in the image storage unit, a color image of which a quantity of color pixels is either a smallest value or a largest value as an optimal color image and configured to cause the selected color image to be displayed by a predetermined display unit.

7. The ultrasound diagnosis apparatus according to claim 1, further comprising:
a filter unit configured to set a frequency hand being an elimination target based on the reception signal received by the ultrasound probe and to eliminate the frequency band from the reception signal; and
an image storage unit configured to store therein a plurality of color images that are generated by the image generating unit for a mutually same image taking position from a reflected-wave signal that passed the filter unit, under the control of the controlling unit, wherein
the controlling unit includes: an image selecting unit configured to select, from among the plurality of color images that are generated for mutually the same image taking position and are stored in the image storage unit, a color image of which an upper-limit frequency of the elimination target frequency band that is set by the filter unit and is used in the generating process performed by the image generating unit is lowest as an optimal color image and configured to cause the selected color image to be displayed by a predetermined display unit.

8. The ultrasound diagnosis apparatus according to claim 1, wherein the controlling unit controls the image generating unit so as to generate the color image, while an ultrasound wave transmitting process performed by the ultrasound probe is being stopped.

9. The ultrasound diagnosis apparatus according to claim 1, wherein the gain selecting unit sequentially extracts the index values of two color images corresponding to two gains, starting from a smallest gain among two or more of the gains and, when a change amount between the two extracted index values becomes equal to or larger than a change-amount threshold value, the gain selecting unit selects the smaller of the two gains as an optimal gain.

10. The ultrasound diagnosis apparatus according to claim 1, wherein the gain selecting unit calculates a change amount between the index values corresponding to two gains, for each set made up of two adjacent gains among two or more of the gains, and selects a gain that approximates to the two gains by which the calculated change amount is largest, as an optimal gain.

11. The ultrasound diagnosis apparatus according to claim 6, further comprising: a quantifying unit configured to calculate a ratio between the quantity of color pixels in the color image and a quantity of pixels in the color image, as quantitative information that quantitatively indicates movement information contained in the color image selected by the image selecting unit.

12. The ultrasound diagnosis apparatus according to claim 7, further comprising: a quantifying unit configured to calculate a ratio between the quantity of color pixels in the color image and a quantity of pixels in the color image, as quantitative information that quantitatively indicates movement information contained in the color image selected by the image selecting unit.

13. An image generating method comprising:
obtaining movement information by adjusting a reception signal received by an ultrasound probe, by using a gain;
controlling an image generating unit configured to generate a color image in which color pixels are allocated based on the movement information;
calculating an index value indicating a distribution of the color pixels, based on two or more of the color images;
selecting a display gain by comparing the index values calculated with respect to two or more of the gains; and
controlling the image generating unit so as to generate a color image in which color pixels are allocated based on the display gain and the movement information obtained from the reception signal.

14. An image processing apparatus comprising:
an index calculating unit configured to calculate an index value indicating a distribution of color pixels, from a plurality of color images in each of which the color pixels are allocated by an image generating unit based on movement information obtained by adjusting a reception signal received by an ultrasound probe by using a gain;
a gain selecting unit configured to select a display gain by comparing the index values calculated with respect to two or more of the gains; and
a controlling unit configured to control the image generating unit so as to generate a color image in which color pixels are allocated based on the display gain and the movement information obtained from the reception signal.
